(19) United States
(12) Patent Application Publication
(10) Pub. No.: US 2011/0060036 A1
(43) Pub. Date: Mar. 10, 2011

(54) Branched Multifunctional Nanoparticle Conjugates And Their Use

(75) Inventors: Shuming Nie, Atlanta, GA (US); Hongwei Duan, Decatur, GA (US); Min Kuang, Decatur, GA (US)

(73) Assignee: EMORY UNIVERSITY, Atlanta, GA (US)

(21) Appl. No.: 12/888,767

(22) Filed: Sep. 23, 2010

Related U.S. Application Data
(63) Continuation of application No. PCT/US2009/038652, filed on Mar. 27, 2009.
(60) Provisional application No. 61/072,220, filed on Mar. 29, 2008.

(51) Int. Cl. A61K 31/337; C07D 305/14; A61P 35/00; B82Y 5/00

(52) U.S. Cl. 514/449; 549/510; 977/773; 977/906

(57) ABSTRACT

Disclosed herein are compounds and compositions including a polyglycerol nanocarrier, a therapeutic agent or imaging agent, and optionally a targeting agent. In certain aspects the disclosed compounds include biocompatible hyperbranched polymer nanocarriers. Such compounds and compositions are useful for the targeted delivery of antitumor agents and imaging agents to tumors in vivo. Methods are also disclosed for detecting and treating such tumors.

### CROSS REFERENCE TO RELATED APPLICATION

This application is a continuation and claims the benefit of PCT/US2009/038652, filed Mar. 27, 2009 and claims the benefit of U.S. Provisional Application No. 61/072,220, filed on Mar. 29, 2008, which is incorporated herein by reference.

### ACKNOWLEDGEMENT OF GOVERNMENT SUPPORT

This invention was made with government support under grant numbers R01 CA108468 and U54 CA119338, awarded by the National Institutes of Health. The government has certain rights in the invention.

### FIELD

The present disclosure concerns drug delivery of therapeutic or imaging agents to a target tissue. In general the disclosed compounds include a targeting component, a therapeutic or imaging component and a nanocarrier component. The disclosure also concerns compositions containing such compounds and methods for using such compounds and compositions.

### BACKGROUND

Considerable pharmaceutical research has been performed to discover systems that selectively deliver a pharmaceutical agent to a desired anatomic location, such as a site in need of treatment. In spite of some progress in this area, many pharmaceutical treatments still impart substantial risk to the patient due to lack of selective drug delivery. The risks are particularly acute in cancer therapy because pharmacologically active anticancer drugs are often quite toxic and reach tumor tissue with poor specificity and dose-limiting toxicity.

Anti-cancer drugs which are effective in attacking malignant cells to destroy them, or at least limit their proliferation, generally affect benign cells as well. Although it is desirable to concentrate a cytotoxic agent at a targeted site, current cancer treatment protocols instead involve non-specific or systemic dosing, with careful monitoring of the patient. In view of the narrow therapeutic index, the dose is selected to be just below the amount that will produce acute (and sometimes chronic) toxicity that can lead to life-threatening cardiomyopathy, myelotoxicity, hepatic toxicity, or renal toxicity. Alopecia (hair loss), mucositis, stomatitis, and nausea are other common, but generally not life-threatening, side effects at these doses.

Previous attempts to administer cytotoxic drugs by direct injection into the location of the organ having the malignancy have been only partially effective because of dispersion of the drug from the target location. Such dispersion cannot be totally avoided, hence excessive quantities of drug need to be administered to attain a desired result. Although careful clinical monitoring helps minimize extensive damage or loss of viable non-targeted tissue, a compound that is actively transported through standard biological systems to the treatment site prior to activation of the cytotoxic agent would be very useful. Thus, there exists a need for a drug delivery system that can accomplish site-specific release of a therapeutic agent in target cells, tissues, or organs.

Efforts have been made in the past to use nanotechnology to achieve site-specific delivery of therapeutic and diagnostic agents. Nanomaterials have beneficial optical, magnetic and structural properties that are not available in their bulk and molecular counterparts. Nanoparticles have been specifically directed to tumors through both passive and active targeting approaches by taking advantaging of the tumor-featured enhanced permeability and retention (EPR) effect or molecular recognition of targeting ligands (antibody, peptide and small molecules), respectively. However, efforts to use nanoparticles for targeted delivery have been frustrated by numerous biological barriers. For example, non-specific uptake of nanoparticles by the reticuloendothelial systems (RES) often leads to accumulation of the majority of the delivered nanoparticles in the liver and spleen. This non-specific uptake not only reduces the effect of the nanoparticles for diagnosis and therapeutic uses, but can also lead to unexpected sides effects.

### SUMMARY OF THE DISCLOSURE

Existing problems with targeted nanoparticle delivery are addressed by providing a class of self-assembled nanoparticles containing polyglycerol-drug conjugates as multi-functional nano-platforms. The nanoparticles are suitable for use in medical imaging and therapy. The self-assembled nanoparticles have shown excellent tumor targeting capacity though passive targeting and ligand-directed active targeting while maintaining low non-specific RES uptake.

Polyglycerol (PG) contains ether backbones. In some embodiments, the PEG is conjugated with a hydrophobic species, in which the PG self-assembles into uniform nanoparticles. These nanoparticles avoid rapid renal clearance and give rise to nanoparticles with neutral surfaces, which increases the overall blood circulation of the conjugates. In specific anticancer examples, these nanoparticles enhance the EPR effect of induced preferential accumulation of nanoparticles in tumors.

The disclosed self-assembled nanoparticles have also been found to very efficiently assemble into complexes of individual nanoparticles, and disassemble again under in-vivo conditions, for example in the systemic circulation and in accumulated organs, which facilitates their clearance in non-specific organs such as the RES system. In certain examples, the assembly of the nanoparticles is enhanced when the polyglycerol particle is conjugated to a hydrophobic agent, such as a hydrophobic pharmaceutical agent, such as a therapeutic or imaging agent. In additional examples, the entanglement and aggregation of individual nanoparticles into larger complexes of nanoparticles is promoted by attaching the agent to the nanoparticle with a linker. In certain disclosed examples, the advantageous properties of the nanoparticles provide greatly enhanced efficacy of PG-conjugated therapeutic and diagnostic agents (such as PG-conjugated paclitaxel (TX) compared to free TX in a breast tumor model).

Linking an optical imaging agent, such as a near-infrared dye, to the nanoparticle enables the in-vivo imaging and tracking of the delivery of nanoparticles. The disclosed nanoparticles are therefore useful as imaging agents, and also provide valuable information about the pharmacokinetics and biodistribution of the nanoparticles. The imaging agents assist in the detection of primary and secondary tumors, and promote the understanding of tumor biology under in-vivo conditions.

Compounds are therefore disclosed herein that include a nanocarrier, a therapeutic agent or imaging agent, and a targeting agent. In one embodiment, the nanocarrier is a hyperbranched polyglycerol polymer, which is for example covalently bonded to a therapeutic agent, and imaging agent, or both.

The disclosed compounds and compositions are useful for drug delivery, in particular, selective in vivo drug delivery and localization to tumor tissue preferentially over other tissues. Accordingly, compositions and methods for their use for treating a subject having a proliferative disorder, particularly those characterized by tumor development, are disclosed herein.

Embodiments of the disclosed compounds accomplish selective tissue targeting by using a covalently bound targeting agent to exploit up-regulated receptors to deliver a therapeutic agent to a cancer cell selectively. Certain embodiments of the disclosed compounds exploit another feature of cancerous tissues to provide selective delivery of therapeutic agents or imaging agents to such tissues. For example, embodiments of the compounds and compositions that include a nanoparticle or self-assemble to form a nanoparticle can benefit from the enhanced permeability and retention effect (EPR effect) and accumulate in tumors.

Self-assembled nanoparticles disclosed herein can be used to deliver both compounds that are covalently and noncovalently bound to the nanoparticle. For example, the self-assembled nanoparticles can include, by encapsulation, small molecules, such as targeting agents, imaging agents and/or therapeutic agents.

Thus, in particular embodiments, compositions include a nanocarrier optionally functionalized with a therapeutic agent and/or a targeting agent, and also include a non-covalently associated therapeutic agent. Examples of such compositions include those wherein the composition self-assembles to form a nanoparticle including the nanocarrier and free therapeutic agent.

### BRIEF DESCRIPTION OF THE DRAWINGS

FIG. 1A is a schematic drawing illustrating the production of encapsulated paclitaxel nanoparticles. The drawing illustrates the production of self-assembled nanoparticles including covalently bound highly branched polyglycerol (PG, molecular weight=20,000 Da) and paclitaxel (TX) self-assembled with an imaging agent and free therapeutic agent (free paclitaxel) with a folate (FA) targeting ligand.

FIG. 1B is shows UV-vis and fluorescence spectra of the conjugates, with absorption peaks of TX at 230 nm and FA at 280 nm.

FIG. 1C shows hydrodynamic size distribution of the self-assembled nanoparticles measured by dynamic light scattering, where GFT is a FA-targeted polyglycerol nanoparticle with paclitaxel, and GT is a non-targeted polyglycerol nanoparticle with paclitaxel.

FIG. 1D is a TEM (transmission electron microscopy) photomicrograph of encapsulated nanoparticles formed as illustrated by FIG. 1A.

FIG. 1E is a bar graph illustrating the cytotoxicity of polyglycerol PG (polyglycerol nanoparticle alone), GFT, GT and TX (paclitaxel alone).

FIG. 1F is a photomicrograph showing fluorescence of dye-labeled targeted nanoparticles in MDA-MB-231 cells, illustrating selective uptake of targeted nanoparticles by the cells.

FIG. 2A is a series of photographs showing in vivo fluorescent imaging of tumors in mice at different times following injection of polymer drug conjugates GT and PG labeled with cy55.

FIG. 2B shows the quantitative decline in fluorescence intensity of GFT, GT and PG.

FIG. 2C is a photomicrograph that shows high sensitivity fluorescence images of single dye-tagged nanoparticles in 100% fetal bovine serum (FBS) when a diluted nanoparticle solution was spread on a glass surface.

FIG. 2D is a graph that shows the overall integrated fluorescence intensity measured from the bulk solution of dye-labeled nanoparticles from FIG. 2C.

FIG. 2E is a graph showing the percentage of drug released over time when the PG conjugate is incubated in PBS at pH 5.2 and 7.0 at 37° C.

FIG. 3A is a schematic drawing illustrating the vascular extravasation of the PG conjugates, and their extravascular disassembly, diffusion and active targeting of tumor cells.

FIG. 3B is a schematic drawing illustrating clearance of nanoparticles from the liver, wherein nanoparticle conjugates exit the liver through either the vascular system (blood vessels and lymphatics) or a hepatic pathway (bile ducts).

FIG. 4A is a series of photographs similar to FIG. 2A, but comparing GFT and GT nanoparticle conjugates.

FIGS. 4B-4E illustrate comparative organ distribution of nanoparticles after a single tail-vein injection of GFT and GT.

FIGS. 4F-4H are fluorescent images and graphs that illustrate advantages offered by targeted nanoparticles (GFT) as compared to non-targeted nanoparticles (GT) for barely palpable tumors (about 2-3 mm in diameter).

FIG. 5A is a graph showing tumor growth curves in a MDA-MB-231 tumor with average sizes of 100-120 mm³ following injection of TX, GFT or GT, while FIG. 5B shows the tumor growth curve for the same tumor with average sizes of 30 mm³.

FIG. 5C shows Ki67 biomarker staining on tumor sections and FIG. 5D shows H&E staining of tumor sections of tumors treated with GFT, GT, TX and control PBS saline.

FIG. 6A shows fluorescence spectra of cy5.5 collection from mouse urine, and FIG. 6B shows the spectra from nanoparticles in PBS buffer.

FIG. 7 is a graph that illustrates the variation of fluorescence intensity at tumor sites after nanoparticles are injected in the mouse tail vein.

FIG. 8 is a pair of photographs at 10 minutes and 6 hours after cy5.5 labeled PG (MW=20,000) is injected in a tail vein.

FIG. 9 shows fluorescent imaging of the uptake of GT nanoparticles by mouse macrophage cells (RAW cells).

### DETAILED DESCRIPTION

The following explanations of terms and methods are provided to better describe the present compounds, compositions and methods, and to guide those of ordinary skill in the art in the practice of the present disclosure. It is also to be understood that the terminology used in the disclosure is for the purpose of describing particular embodiments and examples only and is not intended to be limiting.

### ABBREVIATIONS

A: Therapeutic or diagnostic agent

DCC: N,N′-Dicyclohexylcarbodiimide

FA: folate

GFT: FA-targeted polyglycerol nanoparticle with paclitaxel

GT: non-FA targeted polyglycerol nanoparticle with paclitaxel

NHS: N-hydroxysuccinimide

NIR: near infrared

PEG: polyethylene glycol

PG: polyglycerol

RES: reticuloendothelial system

TEM: transmission electron microscopy

TX: taxol or paclitaxel

X: nanocarrier

Y: targeting agent

### TERMS AND METHODS

Ranges can be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint.

In this specification and in the claims which follow, reference will be made to a number of terms which shall be understood to have the following meanings.

“Optional” or “optionally” means that the subsequently described event or circumstance can but need not occur, and that the description includes instances where said event or circumstance occurs and instances where it does not.

The term “antibody” means an immunoglobulin, whether natural or wholly or partially synthetically produced. All derivatives thereof which maintain specific binding ability are also included in the term. The term also covers any protein having a binding domain which is homologous or largely homologous to an immunoglobulin binding domain. These proteins may be derived from natural sources, or partly or wholly synthetically produced. Antibodies used herein may be monoclonal or polyclonal.

The term “antibody fragment” refers to any derivative of an antibody which is less than full-length. In an exemplary embodiment, the antibody fragment retains at least a significant portion of the full-length antibody's specific binding ability. The antibody fragment may optionally be a single chain antibody fragment. Alternatively, the fragment may comprise multiple chains which are linked together, for instance, by disulfide linkages. The fragment may also optionally be a multimolecular complex. A functional antibody fragment will typically comprise at least about 50 amino acids and more typically will comprise at least about 200 amino acids.

“Derivative” refers to a compound or portion of a compound that is derived from or is theoretically derivable from a parent compound.

“Physiologically labile bond” refers to a bond that may be cleaved under physiological conditions (for example metabolically, solvolytically or in another manner). Such bonds are well known in the art and examples are described in Drugs of Today, Volume 19, Number 9, 1983, pp 499-538 and in Topics in Chemistry, Chapter 31, pp 306-316 and in “Design of Prodrugs” by H. Bundgaard, Elsevier, 1985, Chapter 1 (these disclosures are incorporated herein by reference).

As used herein the term “physiological conditions” refers to temperature, pH, ionic strength, viscosity, and like biochemical parameters which are compatible with a viable organism, and/or which typically exist intracellularly in a viable mammalian cell.

As used herein, the term “self-assembled” refers to any non-covalent association of two or more molecules. Typically, self-assembly occurs in an aqueous solvent, such as under physiological conditions. Examples of self-assembled structures include, without limitation, micelles and liposomes. As used herein, self-assembly also refers to the aggregation of individual nanoparticle conjugates into a larger complex of conjugates.

The term “subject” includes both human and veterinary subjects.

The term “taxane” refers to a class of mitotic inhibitor and anti-microtubule agents that includes taxol (originally obtained from the Pacific Yew tree, and related antitumor drugs such as docetaxel, larotaxel, ortataxel, paclitaxel, tesetaxl, and epothilones such as ixabepilone. The taxanes also include such agents that have been derivatived to improve their bioavailability, for example by making them more soluble in aqueous environments, or in oily delivery vehicles.

The term “treating a disease” refers to inhibiting the full development of a disease or condition, for example, in a subject who is at risk for a disease such as a tumor (for example, leukemia or a lymphoma). “Treatment” refers to a therapeutic intervention that ameliorates a sign or symptom of a disease or pathological condition after it has begun to develop. As used herein, the term “ameliorating,” with reference to a disease or pathological condition, refers to any observable beneficial effect of the treatment. The beneficial effect can be evidenced, for example, by a delayed onset of clinical symptoms of the disease in a susceptible subject, a reduction in severity of some or all clinical symptoms of the disease, a slower progression of the disease, a reduction in the number of metastases, an improvement in the overall health or well-being of the subject, or by other parameters well known in the art that are specific to the particular disease. A “prophylactic” treatment is a treatment administered to a subject who does not exhibit signs of a disease or exhibits only early signs for the purpose of decreasing the risk of developing pathology.

“Neoplasia” refers to the process of abnormal and uncontrolled cell growth. Neoplasia is one example of a proliferative disorder. The product of neoplasia is a neoplasm (a tumor), which is an abnormal growth of tissue that results from excessive cell division. A tumor that does not metastasize is referred to as “benign.” A tumor that invades the surrounding tissue and/or can metastasize is referred to as “malignant.” Examples of hematological tumors include leukemias, including acute leukemias (such as acute lymphocytic leukemia, acute myelocytic leukemia, acute myelogenous leukemia and myeloblastic, promyelocytic, myelomonocytic, monocytic and erythroleukemia), chronic leukemias (such as chronic myelocytic (granulocytic) leukemia, chronic myelogenous leukemia, and chronic lymphocytic leukemia), polycythemia vera, lymphoma, Hodgkin's disease, non-Hodgkin's lymphoma (indolent and high grade forms), multiple myeloma, Waldenstrom's macroglobulinemia, heavy chain disease, myelodysplastic syndrome, hairy cell leukemia and myelodysplasia.

Examples of solid tumors, such as sarcomas and carcinomas, include fibrosarcoma, myxosarcoma, liposarcoma, chondrosarcoma, osteogenic sarcoma, and other sarcomas, synovioma, mesothelioma, Ewing's tumor, leiomyosarcoma, rhabdomyosarcoma, colon carcinoma, lymphoid malignancy, pancreatic cancer, breast cancer, lung cancers, ovarian cancer, prostate cancer, hepatocellular carcinoma, squamous cell carcinoma, basal cell carcinoma, adenocarcinoma, sweat gland carcinoma, sebaceous gland carcinoma, papillary carcinoma, papillary adenocarcinomas, medullary carcinoma, bronchogenic carcinoma, renal cell carcinoma, hepatoma, bile duct carcinoma, choriocarcinoma, Wilms' tumor, cervical cancer, testicular tumor, bladder carcinoma, and CNS tumors (such as a glioma, astrocytoma, medulloblastoma, craniopharyogioma, ependymoma, pinealoma, hemangioblastoma, acoustic neuroma, oligodendroglioma, menangioma, melanoma, neuroblastoma and retinoblastoma).

Water soluble polyaminoacids, include, without limitation, polylysine, polyglutamic acid, polyaspartic acid, copolymers of lysine, glutamatic acid and aspartic acid, and the like. Polyaminoacids can include the D-, L-, or both forms of the amino acid. For example, “polyglutamic acid” refers to poly-D-glutamic acid, poly-L-glutamic acid, or poly-D,L-glutamic acid.

Unless otherwise explained, all technical and scientific terms used herein have the same meaning as commonly understood by one of ordinary skill in the art to which this disclosure belongs. The singular terms “a,” “an,” and “the” include plural referents unless context clearly indicates otherwise. It is further to be understood that all base sizes or amino acid sizes, and all molecular weight or molecular mass values, given for nucleic acids or polypeptides are approximate, and are provided for description. Although methods and materials similar or equivalent to those described herein can be used in the practice or testing of this disclosure, suitable methods and materials are described below. The term “comprises” means “includes.”

All publications, patent applications, patents, and other references mentioned herein are incorporated by reference in their entirety. In case of conflict, the present specification, including explanations of terms, will control. In addition, the materials, methods, and examples are illustrative only and not intended to be limiting.

Disclosed herein are conjugates, compounds, compositions and methods for treating particular tissues, particularly hyper-proliferative tissues, selectively. Generally the conjugates include at least three components, namely a chemotherapeutic agent or an imaging agent a nanocarrier that is a nanoparticle, an organic polymer or both; and a targeting agent. The three components can be covalently or non-covalently associated.

In one embodiment, the components of the disclosed conjugates are covalently linked to form nanocarrier conjugate compounds. Examples of such compounds include ternary or tripartite molecules including at least one nanocarrier, targeting agent and therapeutic agent. In one embodiment the disclosed compounds have one of the following formulas:

A-X-Y

X-A-Y

or

X-Y-A

With respect to the general formulas, A represents a chemotherapeutic agent or an imaging agent; X represents a nanocarrier that is a nanoparticle, an organic polymer or both; and Y represents a targeting agent. In the specific disclosed embodiments, X is a polyglycerol nanoparticle, and particularly a highly branched polyglycerol nanoparticle. In particular embodiments, X is a polyglycerol homopolymer, such as a hyperbranched polyglycerol homopolymer, that does not include any non-polyglycerol co-polymer. Higher order compounds also are disclosed, including quaternary conjugate compounds.

Reference will now be made in detail to the presently preferred embodiments of the disclosed conjugates compounds, compositions and methods.

### I. NANOCARRIERS

The nanocarrier component of the conjugates and compounds disclosed herein can function to present a multivalent display of the therapeutic agent, the targeting agent, or both. In another aspect, the nanocarrier component may confer sufficient size upon the compounds that the compounds benefit from the EPR effect.

Typically, “nanocarrier” refers to a nanoparticle having a diameter of less than about 1500 nanometers or an organic polymer having a molecular weight of more than about 1000 daltons. As used herein the term “nanoparticle” refers to a particle having a diameter of from about 1 nanometer to about 1500 nanometers. Exemplary types of nanoparticles include, without limitation, colloidal and non-colloidal metal clusters and polymeric nanoparticles, such as micelles, liposomes and oil-in-water emulsions. Typically, the nanoparticles employed herein range in diameter from about 1 nanometer to about 1200 nanometers and more typically from about 10 to about 400 nanometers, such as from about 100 to about 150 nanometers. However, certain nanoparticles, for example metal clusters, such as gold clusters can have a diameter as small as about 0.7 nanometers. In certain embodiments a nanoparticle size of less than 400 nanometers, and even less than 150 nanometers, such as about 100 nanometers provides the cell targeting for the present compounds.

A particular disclosed nanoparticle suitable for use as the nanocarrier component is a polyglycerol (PG) organic polymer, the polymer self-assembles under physiological conditions to form a self-assembled nanoparticle that can include a plurality or aggregation of the organic polymer-containing molecules described herein. However, such self-assembled nanoparticles also can be formed under non-physiological conditions provided that the nanoparticles do not disassemble under physiological conditions.

In one embodiment, the nanocarrier conjugate compounds include, in addition to a targeting agent, a hydrophobic component, such as a hydrophobic chemotherapeutic or imaging agent. The resulting compounds are amphiphilic, which enhances self-assembly of the conjugate compounds. One embodiment of such an amphiphilic compound having a hydrophobic chemotherapeutic agent is a paclitaxel-functionalized polyglycerol polymer. Another embodiment of such an amphiphilic compound is an epothilone-functionalized polyglycerol polymer. In particular embodiments, these compounds include folate or a folate derivative as the targeting agent. Such amphiphilic compounds are particularly effective at self assembly to form nanoparticles under aqueous and/or physiological conditions. In certain examples and as described herein amphiphilic nanocarriers and nanoparticles formed from these nanocarriers can be used to encapsulate other free molecules, such as small therapeutic molecules, including those described herein, such that the molecules are a part of the nanoparticle.

In exemplary embodiments a free small molecule therapeutic agent self assembles along with an amphiphilic nanocarrier to form a nanoparticle including the nanocarrier and the small molecule therapeutic agent. The amphiphilic nanocarriers can thus be used as drug delivery vehicles for encapsulated small therapeutic agents. The small therapeutic agents encapsulated in this way include polar, hydrophobic, ionic and nonionic therapeutic agents. This encapsulation strategy is particularly useful for therapeutic agents that are poorly soluble under physiological conditions. The encapsulation strategy also is useful for drug delivery of antitumor agents, because as demonstrated herein encapsulation results in localization of antitumor agents to tumors in vivo.

In one embodiment the disclosed compounds effectively act as prodrugs by releasing the therapeutic agent at a particular targeted tissue or cell. For example, a therapeutic agent can be bound to the nanocarrier or targeting agent via a physiologically labile bond, such as an ester bond, that is cleaved at the target. In one embodiment, the conjugate compound is relatively benign, whereas the therapeutic agent is cytotoxic when not included in a conjugate compound.

The nanoparticles and organic polymers suitable for use in the presently disclosed conjugates can be monodisperse or polydisperse. Typically, when the conjugate includes an organic polymer, the organic polymer is polydisperse. Generally suitable examples of polymers have an average molecular weight of at least about 250 daltons, such as at least about 1,000 daltons. Such polymers typically have an average molecular weight of from about 1,000 to about 150,000 daltons. More typically suitable polymers have a molecular weight of from about 5,000 to about 100,000 daltons, such as from about 10,000 to about 50,000 daltons.

In one aspect, the polyglycerol nanocarrier is a hyperbranched polymer, such as a dendrimer. The term hyperbranched polymer is used to refer to polymers that incorporate plural copies of at least one branching monomer unit. For example, many polymers are comprised of monomer units that only have two reacting groups, thus the polymers prepared from such monomers are linear. In contrast, hyperbranched polymers incorporate monomers that have three or more reacting groups and thus result in branched polymers. Hyperbranched polymers may be homopolymers, composed of monomers that all have the potential for branching sites, or can be copolymers of branching monomers (those able to react three or more times) with other branching monomers or with linear monomers (those able to react only two times. The hyperbranched polymer compounds employed herein typically are considered to be biocompatible or pharmaceutically acceptable polymers, such that they are suitable for administration to human and/or veterinary subjects. Certain disclosed embodiments of the polyglycerol polymer (such as the hyperbranched polyglycerol polymer) are homopolymers that contain only repeating glycerol subunits. In other example, the hyperbranched polyglycerol polymer may be a heteropolymer that includes one, two or more other polymer subunits. Examples of hyperbranched polyglycerol polymer compounds are disclosed herein and in Macromolecules 1999, 32, 4240-4246 (polyglycerol) and Biomaterials 2006, 27:5471-5479, both of which references are incorporated herein by reference.

Hyperbranched polymers can be characterized by their degree of polymerization, DPₙ. Degree of polymerization is calculated as the molecular weight of the polymer divided by molecular weight of monomer, for example, for polyglycerol, its monomer molecular weight is 74, a 20,000 molecular weight polyglycerol should have 270 repeating units, which is also its degree of polymerization. Of course, this value is an average result because a polymer composition typically includes polymer molecules having different molecular weights. Typically the hyperbranched polymers employed as nanocarriers in the presently disclosed conjugate compounds have a degree of polymerization DPₙ in the range of from about 3 or 4 to about 300, or 10 to about 100, such as from about 15 to about 85, including from about 20 to about 75 and from about 25 to 50. By way of example, the degree of polymerization can be about or at least 4, 5, 6, 7, 8, 9, 10, 11, 12, 13 or 14, and typically is about or at least 15, 16, 17, 18, 19, 20, 21, 22, 23, 24, 25, 26, 27, 28, 29 or 30. In other examples, the degree of polymerization is from about 30 to about 50, including about or at least 31, 32, 33, 34, 35, 36, 38, 39, 40, 41, 42, 43, 44, 45, 46, 47, 48 and about 49. In still other examples, the degree of polymerization is less than about 85, such as from about 40 to about 85. In specific examples, the degree of polymerization for polyglycerol is about 15, about 22, such as 22.2, about 44, such as about 44.4, or about 83, such as about 83.4.

The hyperbranched structures employed herein also are characterized by their degree of branching (DB). For example, linear structures have a DB of 0, whereas a perfectly branched polymer (a dendrimer) has a DB of 1. Typically the hyperbranched polymers employed in the presently disclosed conjugate compounds have a DB of from about 0.5 to about 0.9, such as from 0.5 to about 0.7, and in particular about 0.53, 0.55, 0.57, 0.59 or 0.66.

The polymeric nanocarrier materials employed herein also can be described by their polydispersity. The compounds may be monodisperse, but more typically, the compounds, such as hyperbranched polymeric compounds, have a polydispersity of greater than one, but less than about 1.5, typically less then about 1.3 and more typically less than about 1.25.

Also contemplated for use herein as nanocarriers are compounds known as dendrimers. Dendrimers are highly branched polymers and oligomers having a well-defined chemical structure. As a general rule, dendrimers include a core, a given number of generations of branches, or arms, and end groups. The generations of arms consist of structural units that are identical for the same generation of arms and which may be identical or different for different generations of arm. The generations of arms extend radially in a geometrical progression from a core. The end groups of a dendrimer from the Nth generation are the end functional groups of the arms of the Nth generation or end generation.

The description of dendrimers given above includes molecules containing symmetrical branching; it also includes molecules containing non-symmetrical branching. Dense star polymers, starburst polymers and rod-shaped dendrimers are included in the dendrimers described herein.

Several hyperbranched compounds or dendrimers can be combined together, via a covalent bond or another type of bonding, by means of their end groups to give bridged species.

The nanocarrier component contains plural functional groups for derivatization with therapeutic agents, imaging agents and targeting agents. This is particularly true when the nanocarrier is a highly branched polymer, such as a hyperbranched polyglycerol polymer. Thus, in one embodiment of the conjugate compounds, the compounds have a formula

Aₘ-X-Yₙ

X-Aₘ-Yₙ

or

X-Yₙ-Aₘ

wherein A, X and Y are as described above and n and m independently are integers from 1 to about 500, such as from 5 to about 150. Typically, n and m are integers of from 1 to about 50. In exemplary embodiments the sum of m and n is from about 10 to about 100, such as about 50. In compounds having n greater than one, the targeting agents can be the same or different. Similarly, in compounds wherein m is greater than one, the therapeutic agents can be the same or different. An exemplary embodiment of a compound disclosed herein has the formula Aₘ-X-Yₙ, wherein X represents polyglycerol, A is a paclitaxel moiety and Y is a folate moiety, for example wherein n and m independently are from 1 to about 50.

In another exemplary embodiment, the disclosed compounds include those having the formula

Aₘ-X-Yₙ

wherein A, X and Y are as described above and m and n independently are from 1 to 10, such as from 1 to 5. In a particular embodiment, m is 2 and n is 1. In this embodiment the A moieties can be the same or different. In another embodiment m is 1 and n is 2 and the Y moieties can be the same or different. In these embodiments, the compounds are quaternary compounds including a nanocarrier, an imaging or therapeutic agent and two targeting agents. Alternatively, quaternary compounds can include a nanocarrier, a targeting agent and two A groups, such as two therapeutic agents, two imaging agents or a therapeutic agent and an imaging agent.

Examples of the disclosed polyglycerol-based conjugate compounds have the formula

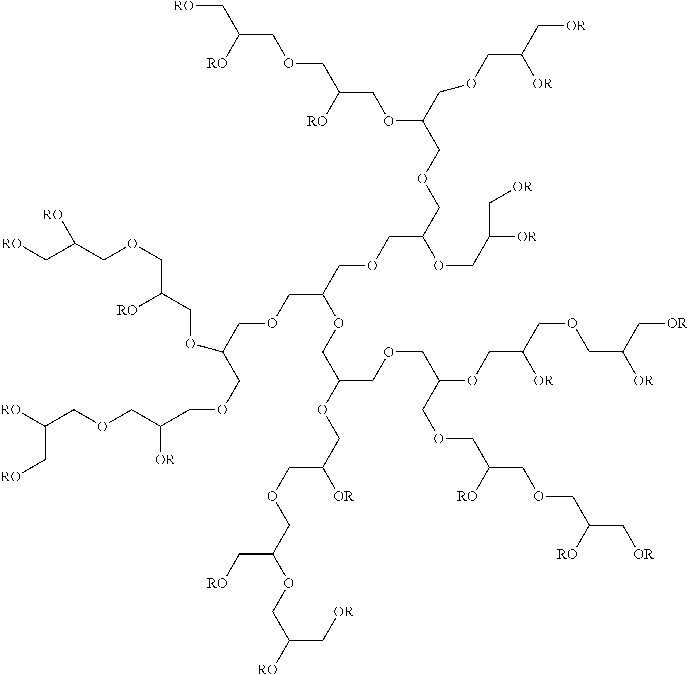

wherein R independently represents for each occurrence H or -GR¹ wherein G is an optional linker group, and R¹ independently for each occurrence represents a therapeutic moiety, such as an antitumor agent or a targeting agent. Although the polyglycerol structure illustrated above is drawn as a single compound, compositions including this compound also typically would include polyglycerol polymers of higher and lower molecular weight, as is known to those of skill in the art. The structure illustrated is exemplary of a class of polyglycerol polymers and of course higher and lower order polyglycerol nanocarriers also can be prepared as is known to those of skill in the art. Moreover, such higher and lower order nanocarrier conjugates are specifically contemplated herein. In particular examples wherein R¹ is an antitumor agent, R¹ represents a taxane, such as docetaxel or paclitaxel, which can be linked, for example through the 2′ taxane position. One particular example of a 2′ linked taxane has the formula

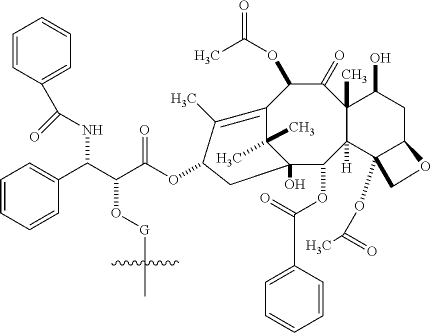

wherein G represents an optional linking group. When present G typically is a bifunctional compound such as an alkyl chain optionally interrupted with one or more heteroatoms. Particular examples of G linker groups are polyethylene glycol (PEG) linkers and short alkyl chains having functional groups to covalently bond functionalities of the components to be linked. In a particular example, a succinate linker was used, so that the formula above could be more specifically represented as

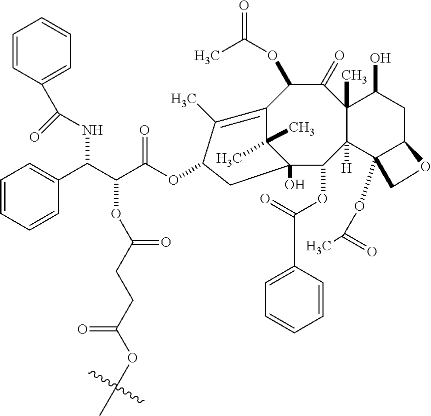

In one embodiment, wherein G is a polyethylene glycol linker and R¹ is an imaging agent -GR¹ has the formula

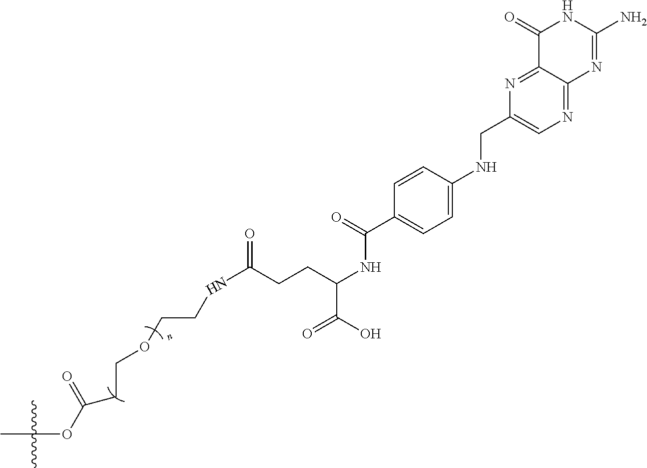

wherein n typically is from 2 to about 150, such as from about 2 to about 10 or from about 8 to about 100, or from 20 to about 50, or in particular examples, 2, 3, 4, 5, 6, 9, 15, 35, 50, 80 or about 110.

Examples of the presently disclosed conjugate compounds, for example ternary and quaternary conjugates, can be represented schematically as

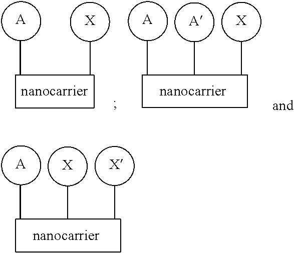

wherein A and X are as described above, A and A′ represent different chemotherapeutic or imaging agents, and X and X′ represent different targeting agents. In one embodiment of a ternary nanocarrier conjugate, the nanocarrier is functionalized with plural copies of A and/or X. Similarly, embodiments of quaternary nanocarrier conjugates are functionalized with plural copies of A, A′, X and/or X′. Certain examples of quaternary nanocarrier conjugates include at least one chemotherapeutic agent and at least one imaging agent.

A variety of methods suitable for preparing examples of the presently disclosed compounds have been developed for assembling and functionalizing metal clusters. These methods have focused on the use of covalent linker molecules that possess functionalities at opposing ends with chemical affinities for the colloids of interest. Examples of this approach are described by Brust et al. Adv. Mater. 1995 7, 795-797, involves the use of gold colloids and well-established thiol adsorption chemistry, Bain & Whitesides, Angew. Chem. Int. Ed. Engl. 1989, 28, 506-512 and Dubois & Nuzzo, Annu Rev. Phys. Chem. 1992, 43, 437-464. U.S. Pat. No. 6,767,702 to Mirkin et al. and U.S. Application Publication No. 2003/0077625, both of which are incorporated herein by reference, disclose metal clusters and methods and reagents for functionalizing such clusters that can be used to prepare the presently disclosed conjugate compounds.

### II. THERAPEUTIC AGENTS AND IMAGING AGENTS

Any therapeutic agent can be used in the disclosed compounds and methods. Appropriate therapeutic agents can be selected based upon the particular tissue or cell type being targeted. That is, the choice of a particular therapeutic agent depends on the particular target molecule or cell and the biological effect it is desired to evoke. For example, in one embodiment the compounds include a targeting agent that targets hyper-proliferative cells, such as cancer cells. In such embodiments the therapeutic agent can be an anti-proliferative agent, including any chemical agent with therapeutic usefulness in the treatment of proliferative diseases or diseases characterized by abnormal cell growth. Such diseases include tumors, neoplasms, and cancer as well as diseases characterized by hyperplastic growth such as psoriasis. In one embodiment, an anti-proliferative agent is an agent of use in treating a lymphoma, leukemia, or another tumor. In one embodiment, an anti-proliferative agent is a radioactive compound. One of skill in the art can readily identify an anti-proliferative agent of use (e.g. see Slapak and Kufe, Principles of Cancer Therapy, Chapter 86 in Harrison's Principles of Internal Medicine, 14th edition; Perry et al., Chemotherapy, Ch. 17 in Abeloff, Clinical Oncology 2nd ed., 2000 Churchill Livingstone, Inc; Baltzer, L., Berkery, R. (eds): Oncology Pocket Guide to Chemotherapy, 2nd ed. St. Louis, Mosby-Year Book, 1995; Fischer, D. S., Knobf, M. F., Durivage, H. J. (eds): The Cancer Chemotherapy Handbook, 4th ed. St. Louis, Mosby-Year Book, 1993).

Classes of useful anti-proliferative agents that can be used in the present compounds include, without limitation, microtubule binding agent, a toxin, a DNA intercalator or cross-linker, a DNA synthesis inhibitor, a DNA and/or RNA transcription inhibitor, an enzyme inhibitor, a gene regulator, enediyne antibiotics and/or an angiogenesis inhibitor. In one embodiment the molecules have sufficient selectivity for a hyper-proliferative tissue such that therapeutic agents having a higher cytotoxicity than is normally acceptable can be used.

“Microtubule binding agent” refers to an agent that interacts with tubulin to stabilize or destabilize microtubule formation thereby inhibiting cell division. Suitable microtubule binding agents include, without limitation, paclitaxel, docetaxel, vinblastine, vindesine, vinorelbine (navelbine), the epothilones, colchicine, dolastatin 15, nocodazole, podophyllotoxin and rhizoxin. Analogs and derivatives of such compounds also can be used and will be known to those of ordinary skill in the art. For example, suitable epothilones and epothilone analogs for incorporation into the present compounds are described in International Publication No. WO 2004/018478, which is incorporated herein by reference. Taxoids, such as paclitaxel and docetaxel are currently believed to be particularly useful as therapeutic agents in the presently disclosed compounds. Examples of additional useful taxoids, including analogs of paclitaxel are taught by U.S. Pat. Nos. 6,610,860 to Holton, 5,530,020 to Gurram et al. and 5,912,264 to Wittman et al. Each of these patents is incorporated herein by reference.

The therapeutic agent may be a cytotoxin which is used to bring about the death of a particular target cell. Exemplary toxins include Pseudomonas exotoxin (PE), ricin, abrin, diphtheria toxin and subunits thereof, ribotoxin, ribonuclease, saporin, as well as botulinum toxins A through F. These toxins are well known in the art and many are readily available from commercial sources (for example, Sigma Chemical Company, St. Louis, Mo.).

One example of a suitable therapeutic agent, diphtheria toxin, is isolated from Corynebacterium diphtheriae. Typically, diphtheria toxin for use in immunotoxins is mutated to reduce or to eliminate non-specific toxicity. A variant known as CRM107, which has full enzymatic activity but markedly reduced non-specific toxicity, has been known since the 1970's (Laird and Groman, J. Virol. 1976, 19, 220), and has been used in human clinical trials. See, U.S. Pat. No. 5,792,458 and U.S. Pat. No. 5,208,021. As used herein, the term “diphtheria toxin” refers as appropriate to native diphtheria toxin or to diphtheria toxin that retains enzymatic activity but which has been modified to reduce non-specific toxicity.

Another exemplary toxin, suitable for use as the therapeutic component of the presently disclosed conjugates is ricin. Ricin is a lectin isolated from Ricinus communis (castor bean). The term “ricin” also references toxic variants thereof. For example, see, U.S. Pat. No. 5,079,163 and U.S. Pat. No. 4,689,401. Ricinus communis agglutinin (RCA) occurs in two forms designated RCA60 and RCA120 according to their molecular weights of approximately 65 and 120 kD, respectively (Nicholson & Blaustein, J. Biochim. Biophys. Acta 1972, 266, 543). The A chain is responsible for inactivating protein synthesis and killing cells. The B chain binds ricin to cell-surface galactose residues and facilitates transport of the A chain into the cytosol (Olsnes et al. Nature 1974, 249, 627-631, and U.S. Pat. No. 3,060,165). Another toxic lectin, abrin, includes toxic lectins from Abrus precatorius. The toxic principles, abrin a, b, c, and d, have a molecular weight of from about 63 and 67 kD and are composed of two disulfide-linked polypeptide chains A and B. The A chain inhibits protein synthesis; the B-chain (abrin-b) binds to D-galactose residues (see, Funatsu, et al. Agr. Biol. Chem. 1988, 52, 1095; and Olsnes, Methods Enzymol. 1978, 50, 330-335,).

In one embodiment, a toxin used to terminate a targeted cell is Pseudomonas exotoxin (PE). Native Pseudomonas exotoxin A (“PE”) is an extremely active monomeric protein (molecular weight 66 kD), secreted by Pseudomonas aeruginosa, which inhibits protein synthesis in eukaryotic cells. The native PE sequence and the sequence of modified PE is provided in U.S. Pat. No. 5,602,095, which is incorporated herein by reference. The method of action of PE is inactivation of the ADP-ribosylation of elongation factor 2 (EF-2). The exotoxin contains three structural domains that act in concert to cause cytotoxicity. Domain Ia (amino acids 1-252) mediates cell binding. Domain II (amino acids 253-364) is responsible for translocation into the cytosol and domain III (amino acids 400-613) mediates ADP ribosylation of elongation factor 2. The function of domain Ib (amino acids 365-399) remains undefined, although a large part of it, amino acids 365-380, can be deleted without loss of cytotoxicity. See, Siegall et al. J. Biol. Chem. 1989, 264, 14256-14261.

The term “Pseudomonas exotoxin” (“PE”) as used herein refers as appropriate to a full-length native (naturally occurring) PE or to a PE that has been modified. Such modifications may include, but are not limited to, elimination of domain Ia, various amino acid deletions in domains Ib, II and III, single amino acid substitutions and the addition of one or more sequences at the carboxyl terminus. See, Siegall et al., supra. In several examples, the cytotoxic fragment of PE retains at least 50%, preferably 75%, more preferably at least 90%, and most preferably 95% of the cytotoxicity of native PE. In one embodiment, the cytotoxic fragment is more toxic than native PE.

Thus, the PE used in the targeted conjugates disclosed herein includes the native sequence, cytotoxic fragments of the native sequence, and conservatively modified variants of native PE and its cytotoxic fragments. Cytotoxic fragments of PE include those which are cytotoxic with or without subsequent proteolytic or other processing in the target cell (e.g., as a protein or pre-protein). Cytotoxic fragments of PE known in the art include PE40, PE38, and PE35.

In several embodiments, the PE has been modified to reduce or eliminate non-specific cell binding, typically by deleting domain Ia, as taught in U.S. Pat. No. 4,892,827, although this can also be achieved, for example, by mutating certain residues of domain Ia. U.S. Pat. No. 5,512,658, for instance, discloses that a mutated PE in which Domain Ia is present but in which the basic residues of domain Ia at positions 57, 246, 247, and 249 are replaced with acidic residues (glutamic acid, or “E”) exhibits greatly diminished non-specific cytotoxicity. This mutant form of PE is sometimes referred to as PE4E.

PE40 is a truncated derivative of PE (see, Pai et al. Proc. Nat'l Acad. Sci. USA 1991, 88, 3358-62; and Kondo et al. J. Biol. Chem. 1988, 263, 9470-9475). PE35 is a 35 kD carboxyl-terminal fragment of PE in which amino acid residues 1-279 have deleted and the molecule commences with a met at position 280 followed by amino acids 281-364 and 381-613 of native PE. PE35 and PE40 are disclosed, for example, in U.S. Pat. No. 5,602,095 and U.S. Pat. No. 4,892,827. In some embodiments, the cytotoxic fragment PE38 is employed. PE38 is a truncated PE pro-protein composed of amino acids 253-364 and 381-613 of which is activated to its cytotoxic form upon processing within a cell (see e.g., U.S. Pat. No. 5,608,039, and Pastan et al., Biochim. Biophys. Acta 1333:C1-C6, 1997). While in some embodiments, the PE is PE4E, PE40, or PE38, any form of PE in which non-specific cytotoxicity has been eliminated or reduced to levels in which significant toxicity to non-targeted cells does not occur can be used in the immunotoxins disclosed herein so long as it remains capable of translocation and EF-2 ribosylation in a targeted cell.

Conservatively modified variants of PE or cytotoxic fragments thereof have at least 80% sequence similarity, preferably at least 85% sequence similarity, more preferably at least 90% sequence similarity, and most preferably at least 95% sequence similarity at the amino acid level, with the PE of interest, such as PE38.

Ribonucleases also can be used as toxins in the disclosed, targeted conjugate compounds (see, Suzuki et al. Nat. Biotech. 1999, 17, 265-70). Exemplary ribotoxins such as α-sarcin and restrictocin are discussed in, for example, Rathore et al. Gene 1997, 190, 31-35; and Goyal and Batra, Biochem. 2000, 345 Pt 2 247-54.

DNA intercalators and cross-linking agents that can be incorporated into the disclosed compounds include, without limitation, cisplatin, carboplatin, oxaliplatin, mitomycins, such as mitomycin C, bleomycin, chlorambucil, cyclophosphamide and derivatives and analogs thereof.

DNA synthesis inhibitors suitable for use as therapeutic agents include, without limitation, methotrexate, 5-fluoro-5′-deoxyuridine, 5-fluorouracil and analogs thereof.

Examples of suitable enzyme inhibitors for use in the presently disclosed conjugates include, without limitation, camptothecin, etoposide, formestane, trichostatin and derivatives and analogs thereof.

Suitable therapeutics that affect gene regulation include agents that result in increased or decreased expression of one or more genes, such as, without limitation, raloxifene, 5-azacytidine, 5-aza-2′-deoxycytidine, tamoxifen, 4-hydroxytamoxifen, mifepristone and derivatives and analogs thereof.

Suitable enediyne antibiotics for incorporation into the disclosed conjugates include naturally occurring enediyne-containing compounds, non-natural compounds and derivatives thereof. Naturally-occurring enediyne antibiotics, including dynemicin (Konishi, et al. J. Chem. Soc. 1990, 112, 3715-3716, esperamicin (Golik et al. J. Amer. Chem. Soc. 1987 109, 3462-3464) and calicheamicin (Lee et al. J. Amer. Chem. Soc. 1987, 109, 3464-3466) are very potent cytotoxins, with IC₅₀ values for inhibition of growth of tumor cell cultures in the low picomolar range. Examples of non-natural enediyne antibiotic compounds suitable for incorporation into the presently disclosed conjugates are disclosed by Nicolaou, K. C. et al, Science 1992, 256, 1172-1178; Nicolaou et al. Proc. Natl. Acad. Sci. USA. 1993, 90, 5881-5888; J. Amer. Chem. Soc. 1992, 114, 8890-8907; J. Amer. Chem. Soc. 1993, 115, 7944-7953; J. Amer. Chem. Soc. 1992, 114, 8908-8921; Wender et al. J. Org. Chem. 1993, 58, 5867-5869; Synthesis, 1994, 1279-1282; U.S. Pat. Nos. 6,124,310 to Denny et al. and 6,514,995 to Zaleski and Rawat. Each of these disclosures is incorporated herein by reference.

Suitable DNA and/or RNA transcription regulators, including, without limitation, actinomycin D, daunorubicin, doxorubicin and derivatives and analogs thereof also are suitable for use in the presently disclosed compounds.

The term “angiogenesis inhibitor” is used herein, to mean a molecule including, but not limited to, biomolecules, such as peptides, proteins, enzymes, polysaccharides, oligonucleotides, DNA, RNA, recombinant vectors, and small molecules that function to inhibit blood vessel growth. Angiogenesis is implicated in certain pathological processes, such as those involved in disorders such as diabetic retinopathy, chronic inflammatory diseases, rheumatoid arthritis, dermatitis, psoriasis, stomach ulcers, and most types of human solid tumors.

Angiogenesis inhibitors are known in the art and examples of suitable angiogenesis inhibitors include, without limitation, angiostatin K 1-3, staurosporine, genistein, fumagillin, medroxyprogesterone, suramin, interferon-alpha, metalloproteinase inhibitors, platelet factor 4, somatostatin, thromobospondin, endostatin, thalidomide, and derivatives and analogs thereof.

Other therapeutic agents, particularly anti-tumor agents, that may or may not fall under one or more of the classifications above, also are suitable for incorporation into the presently disclosed compounds. By way of example, such agents include adriamycin, apigenin, rapamycin, zebularine, cimetidine, and derivatives and analogs thereof.

In certain embodiments, the compounds are targeted to atherosclerotic lesions. In such embodiments the therapeutic agent is effective to reduce or prevent lipid accumulation by the vessel, to increase plaque stability of an atherosclerotic lesion, to inhibit atherosclerotic lesion formation or development, or to induce atherosclerotic lesion regression. Examples of suitable therapeutic agents include those taught by U.S. Pat. No. 6,734,208, to Grainger et al., which is incorporated herein by reference.

Additional suitable therapeutic agents include anti-sense oligonucleotides and the like; biological response modifiers such as muramylpeptides; antifungal agents such as ketoconazole, nystatin, griseofulvin, flucytosine, miconazole or amphotericin B; hormones or hormone analogues such as growth hormone, melanocyte stimulating hormone, estradiol, beclomethasone dipropionate, betamethasone, cortisone acetate, dexamethasone, flunisolide, hydrocortisone, methylprednisolone, paramethasone acetate, prednisolone, prednisone, triamcinolone or fludrocortisone acetate; vitamins such as cyanocobalamin or retinoids; enzymes such as alkaline phosphatase or manganese superoxide dismutase; antiallergic agents such as amelexanox; inhibitors of tissue factor such as monoclonal antibodies and Fab fragments thereof, synthetic peptides, nonpeptides and compounds downregulating tissue factor expression; inhibitors of platelets such as, GPIa, GPIb and GPIIb-IIIa, ADP receptors, thrombin receptors, von Willebrand factor, prostaglandins, aspirin, ticlopidin, clopigogrel and reopro; circulatory drugs such as propranolol; metabolic potentiators such as glutathione; antituberculars such as p-aminosalicylic acid, isoniazid, capreomycin sulfate, cyclosexine, ethambutol, ethionamide, pyrazinamide, rifampin or streptomycin sulfate; antivirals such as acyclovir, amantadine, azidothymidine, ribavirin or vidarabine; blood vessel dilating agents such as diltiazem, nifedipine, verapamil, erythritol tetranitrate, isosorbide dinitrate, nitroglycerin or pentaerythritol tetranitrate; antibiotics such as dapsone, chloramphenicol, neomycin, cefaclor, cefadroxil, cephalexin, cephradine, erythromycin, clindamycin, lincomycin, amoxicillin, ampicillin, bacampicillin, carbenicillin, dicloxacillin, cyclacillin, picloxacillin, hetacillin, methicillin, nafcillin, penicillin, polymyxin or tetracycline; antiinflammatories such as diflunisal, ibuprofen, indomethacin, meclefenamate, mefenamic acid, naproxen, phenylbutazone, piroxicam, tolmetin, aspirin or salicylates; antiprotozoans such as chloroquine, metronidazole, quinine or meglumine antimonate; antirheumatics such as penicillamine; narcotics such as paregoric; opiates such as codeine, morphine and analogs thereof; cardiac glycosides such as deslaneside, digitoxin, digoxin, digitalin or digitalis; neuromuscular blockers such as atracurium mesylate, gallamine triethiodide, hexafluorenium bromide, metocurine iodide, pancuronium bromide, succinylcholine chloride, tubocurarine chloride or vecuronium bromide; sedatives such as amobarbital, amobarbital sodium, apropbarbital, butabarbital sodium, chloral hydrate, ethchlorvynol, ethinamate, flurazepam hydrochloride, glutethimide, methotrimeprazine hydrochloride, methyprylon, midazolam hydrochloride, paraldehyde, pentobarbital, secobarbital sodium, talbutal, temazepam or triazolam.

Additional therapeutic agents may be selected for incorporation into the disclosed compounds as will be apparent to those of ordinary skill in the art upon review of the present specification.

In one embodiment, the compounds disclosed herein can include an imaging agent. As used herein, the term “imaging agent” refers to compounds that can be detected. Examples of imaging agents include magnetic resonance imaging contrast agents, computed tomography (CT scan) imaging agents, optical imaging agents and radioisotopes. In certain compounds according to this embodiment, an imaging agent optionally may be used in place of a therapeutic agent. Thus, the presently disclosed compounds can be used to image a targeted tissue selectively.

Particular examples of suitable imaging agents include, without limitation gadolinium chelating agents, such as gadolinium-DTPA (Gd-DTPA), CT scan imaging agents, such as those including a heavy metal such as iron chelates; near-infrared optical imaging agents, such as Cy 5.5, indocyanine green (ICG) and its derivatives, and the radionuclides indium-111, technetium-99m, yttrium-90 and holmium-166. Additionally, positron emission tomography (PET) may be possible using positron emitters of oxygen, nitrogen, iron, carbon, or gallium.

In one embodiment, the disclosed compounds include those having both a therapeutic agent and an imaging agent. One example of such compounds has the formula

Aₘ-X-Yₙ

wherein m is 2 or greater, such as from 2 to about 500, such as from 5 to about 150. Typically m is from 2 to about 50, such as from 2 to about 10. Examples of such compounds can include a single imaging agent and a plurality of therapeutic agents or a single therapeutic agent and a plurality of imaging agents. However, in one embodiment, m is 2 and the formula includes a single therapeutic agent and a single imaging agent.

### III. TARGETING AGENTS

The targeting agent can be any ligand moiety, such as an antibody, growth factors, cytokines, cell adhesion molecules, their receptors, peptide, protein or small molecule, such as a receptor agonist, antagonist or enzyme inhibitor that binds to a cell, typically a particular cellular receptor. It is understood that when a particular targeting agent is referred to, fragments, residues and derivatives thereof also are intended.

In one embodiment, the compounds include plural targeting agents, which can be the same or different. For example, a compound can present an effectively multivalent display of plural targeting agents, to enhance affinity, avidity or selectivity of a nanocarrier therapeutic. Alternatively, the disclosed compounds can include different targeting agents to, for example target different cell-types or tissues. Specifically, the compounds include those of the formula

Aₘ-X-Yₙ

wherein A, X, Y and m are as described above, and n is from 2 to about 500, such as from 5 to about 150. Such compounds include those having n be an integer of from 2 to about 50, such as from 2 to about 10. In one embodiment of such a compound, n is two and the formula includes two different Y targeting moieties.

Both conventional and genetically engineered antibodies may be employed as targeting agents. The use of human antibodies may be preferred to avoid possible immune reactions. In one embodiment, the targeting agent is an antibody that binds to a member of the human epidermal growth factor receptor (EGFR) family. The human EGFR family includes EGFR-1 (HER-1), EGFR-2 (HER-2), EGFR-3 (HER-3) and EGFR 4 (HER-4). EGFR expression has been documented extensively in a wide variety of malignant tumors including lung, head and neck, colon, breast, and prostate, etc.⁵⁰⁻⁵⁶ Several studies, have demonstrated that overexpression of EGFR correlates with reduced overall survival, increased risk of disease recurrence and metastasis. (See, for example, Grandis J R, Melhem M F, Gooding W E, et al. Levels of TGF-α and EGFR protein in head and neck squamous cell carcinoma and patient survival. J Natl Cancer Inst 1998, 90:824-32; Mauizi M, Almadori G, Ferrandina G, et al. Prognostic significance of epidermal growth factor receptor in laryngeal squamous cell carcinoma. Br J Cancer 1996, 74:1253-7; Yamanaka Y, Friess H, Kobrin M S, Buchlen M, Beger H G, Korc M. Coexpression of epidermal growth factor receptor and ligands in human pancreatic cancer is associated with enhanced tumor aggressiveness. Anticancer Res 1993, 13:565-70; Neal D E, Sharples L, Smith K, Fennelly J, Hall R R, Hams A L. The epidermal growth factor receptor and the prognosis of bladder cancer. Cancer 1993, 65: 1619-25.) Overexpression of EGFR also correlates to a poor response to therapeutic agents. See, Aziz S A, Pervez S, Khan S, Kayani N, Rahbar M H. Epidermal growth factor receptor (EGFR) as a prognostic marker: an immunohistochemical study on 315 consecutive breast carcinoma patients. J Pak Med Assoc 2002, 52:104-10; Tsutsui S, Ohno S, Murakami S, Hachitanda Y, Oda S. Prognostic value of epidermal growth factor receptor (EGFR) and its relationship to the estrogen receptor status in 1029 patients with breast cancer. Breast Cancer Res Treat 2002, 71:67-75; Nicholson R I, Gee J M, Harper M E. EGFR and cancer prognosis. Eur J Cancer 2001, 37 Suppl 4:S9-15.

In a particular embodiment, a single chain EGFR antibody (ScFv EGFR) is used as a targeting agent for the formulation of nanocarrier conjugates disclosed herein.

In one embodiment, the targeting agent is a ligand for a cell surface receptor. In one aspect of this embodiment the targeting agent induces receptor-mediated endocytosis, such as potocytosis. Examples of suitable targeting agents that may induce receptor-mediated endocytosis include, without limitation, folate, insulin, nerve growth factor, luteinizing hormone, calcitonin and catecholamines.

In one embodiment of the compounds disclosed herein, the targeting agent selected binds to a receptor that is present at higher density on the targeted cells. For example, certain tumor cells over-express receptors involved in the uptake of folate, biotin and/or vitamins, such as vitamin B₁₂. Other receptors that can be targeted on tumor cells include, without limitation, transferrin receptor, mucins, multiple P-glycoprotein, cathepsin B and CD44. Accordingly, disclosed herein are compounds that employ antibodies directed to the receptors recited above, as well as compounds that employ small molecule targeting agents, such as folate, vitamin B₁₂ and/or biotin, and derivatives thereof as targeting agents to direct therapeutic agents to such cells. The folate receptor, for example, is known to be overexpressed on the surface of cancer cells in the case of epithelial malignancies, such as ovarian, colorectal, and breast cancer, whereas in most normal tissue it is expressed in very low levels. See, Leamon and Reddy Adv. Drug Deliv. Rev. 2004, 56, 1127-1141; Lee and Low J. Biol. Chem. 1994, 269, 3198-3204. Embodiments of targeting agents directed to the folate receptor include, without limitation, folic acid, folic acid derivatives and analogs, antifolates and deazafolates. As used herein, the term “folate” shall include all such structures. Examples of such folates include folic acid, dihydrofolic acid, tetrahydrofolic acid, folinic acid, pteropolyglutamic acid, dihydrofolates, tetrahydrofolates, tetrahydropterins, 1-deaza, 3-deaza, 5-deaza, 8-deaza, 10-deaza, 1,5-dideaza, 5,10-dideaza, 8,10-dideaza and 5,8-dideaza folate analogs, and antifolates.

In one embodiment a peptide targeting agent is selected using combinatorial techniques, such as phage display (see, U.S. Pat. No. 5,223,409) or variations of phage display, with which those of ordinary skill in the art will be familiar. Similarly, small molecule ligands, such as receptor agonists, antagonists and inhibitors, suitable for use as targeting agents can be prepared and selected using combinatorial techniques.

Cytokines, growth factors and peptide hormones that can be used as the targeting component in the presently disclosed compounds include epidermal growth factor, nerve growth factor, somatostatin, endothelin, interleukin-1, interleukin-2, tumor necrosis factor, parathyroid hormone, insulin like growth factor I and fragments thereof.

In one embodiment the disclosed compounds employ anti-angiogenic factors as targeting agents, such as, interferon-α, interferon-γ, thrombospondin, angiogenin, bradykinin, basic fibroblast growth factor, fibrin, fibrinogen, histamine, nicotinamide, platelet activating factor, prostaglandins, spermine, substance P, transforming growth factor-α, transforming growth factor-β, vitronectin and fragments thereof. Targeting agents also can be selected to target atherosclerotic lesions, for example, annexin V atherosclerotic plaque binding peptides such as YRALVDTLK, YAKFRETLEDTRDRMY and RALVDTEFKVKQEAGAK, can be used to target such lesions. Additional targeting agents that can be used target receptors associated with angiogenesis but may not be angiogenetic factors, such agents include, without limitation, antibodies, angiopoietin, α₂-antiplasmin, endosialin, hepatocyte growth factor, leukemia inhibitory factor, RGD-peptides, such as cyclic RGD_{D}FV, placental growth factor, selecting, pleiotropin, thymidine phosphorylase, tumor growth factor, sialyl Lewis X, osteopontin, syndecan, tissue factor, VCAM, vascular endothelial growth factor related protein, vascular endothelial growth factor-A receptor, von Willebrand factor-related antigen and fragments thereof.

Additional over-expressed receptors that can be targeted by using their ligands in the presently disclosed compounds and compositions are known to those of ordinary skill in the art.

### IV. CONJUGATION CHEMISTRY

Numerous methods and reagents for coupling the components of the presently disclosed conjugates are well known to those of ordinary skill in the art. Table 1 lists representative suitable functional groups that may be present on a polymer, nanoparticle, therapeutic agent, imaging agent, targeting agent or linker and can be used to couple these materials.

TABLE 1
Functional Group Pairs for Conjugation Chemistry

| Functional Groups: | Reacts With: |
|---|---|
| ketone groups (such as aldehydes) | amino, hydrazido and aminooxy |
| Imide | amino, hydrazido and aminooxy |
| Cyano | hydroxy |
| alkylating agents (such as haloalkyl | thiol, amino, hydrazido, |
| groups and maleimide derivatives) | aminooxy |
| carboxyl groups (including activated | amino, hydroxy, hydrazido, |
| carboxyl groups) | aminooxy |
| activated sulfonyl groups (such as | amino, hydroxy, hydrazido, |
| sulfonyl chlorides) | aminooxy |
| sulfhydryl | sulfhydryl |
| His-tag (such as a 6-His tagged peptide | nickel nitriloacetic acid |
| or protein) | |

U.S. Pat. No. 6,303,752 to Olsen et al., which is incorporated herein by reference, describes use of the functional groups listed in Table 1 among others to couple linkers, polymers and proteins.

Other reagents than the exemplary coupling partners in Table 1 can be used to couple components of the compounds disclosed herein. For example, azide-containing compounds can be coupled to other molecules via Staudinger ligation. Suitable reagents for Staudinger ligation can be prepared according to the methods disclosed by Saxon and Bertozzi in U.S. Pat. No. 6,570,040 and by Raines et al. in U.S. Patent Publication No. 20040087779. The '040 patent and the '779 publication are incorporated herein by reference in their entireties.

In particular, the coupling methods suitable for assembling the disclosed conjugates include, without limitation, amino-reactive acylating agents, such as isocyanates and isothiocyanates, which form stable urea and thiourea derivatives respectively. Examples of such compounds have been used for protein crosslinking as described by Schick, A. F. et al. in J. Biol. Chem. 1961 236, 2477. Active esters are particularly useful for preparing the disclosed conjugate compounds, such as nitrophenylesters or N-hydroxysuccinimidyl esters. Suitable reagents and conditions for acylating amino groups using active esters are described by Bodanszky, M. and Bodanszky, A.; The Practice of Peptide Synthesis; Springer Verlag, New York, 1994; and by Jones, J.; Amino Acid and Peptide Synthesis; 2nd ed.; Oxford University Press, 2002, both of which are incorporated herein by reference.

Other suitable linkages formed using the reagents listed in Table 1 include disulfide linkages, formed by the oxidative coupling of two sulfhydryl-containing molecules. Another exemplary coupling technique employs a chelated nickel moiety, such as nickel nitriloacetic acid, which couples with His-tagged peptides and proteins, including His-tagged antibodies.

Peptides and proteins, including antibodies, also can be covalently coupled to a nanocarrier. For example, a native chemical ligation technique, such as described by Kent et al. in Chemical protein synthesis by solid phase ligation of unprotected peptide segments. J. Am. Chem. Soc. 121, 8720-27 (1999), can be used, as can the Staudinger ligation protocols discussed above.

Additional techniques for coupling materials, including those having the functional groups listed in Table 1, are taught by R. F. Taylor, (1991), “Protein immobilisation. Fundamental and applications”, Marcel Dekker, N.Y.; S. S. Wong, (1992), “Chemistry of Protein Conjugation and Crosslinking”, CRC Press, Boca Raton; and G. T. Hermanson et al., (1993), “Immobilized Affinity Ligand Techniques”, Academic Press, N.Y. Each of these publications is incorporated herein by reference.

In some embodiments the components of the nanocarrier compounds are directly bonded together without use of a spacer or linker component. For example, a therapeutic agent, an imaging agent and/or a targeting agent is directly bonded to the nanocarrier. However, in certain embodiments coupling of the conjugates disclosed herein include a linker covalently or non-covalently linking at least one of a therapeutic agent to the nanocarrier, the nanocarrier to a targeting agent, and a therapeutic agent to a targeting agent. In particular examples, the linker forms a covalent linkage between these agents, and thus comprises two or more reactive moieties, e.g. as described above, connected by a spacer element. The presence of such a spacer permits bifunctional linkers to react with specific functional groups within a molecule or between two different molecules, resulting in a bond between these two components and introducing extrinsic linker-derived material into the conjugate. The reactive moieties in a linking agent may be the same (homobifunctional agents) or different (heterobifunctional agents or, where several dissimilar reactive moieties are present, heteromultifunctional agents), providing a diversity of potential reagents that may bring about covalent bonding between any chemical species, either intramolecularly or intermolecularly.

Linkers can enhance self-assembly of the nanoparticles. Linkers of particular lengths are particularly advantageous in promoting the entanglement of individual nanoparticles into larger complexes of nanoparticles. Examples of useful linkers include (1) carboxylic acids from 2 to 10 carbons (for example, the linker derived from succinic anhydride that has 4 carbons), (2) amino acids (from one to 10 amino acids in length), (3) polyethylene glycols in the molecular weight range of 500 Daltons to 20 KDaltons.

Exemplary linker moieties used to connect the components of the conjugates disclosed herein include oligomer or polymer moieties such as polyalkylene oxides. Suitable examples of such polymers include polyethylene glycols which are substantially non-antigenic. Also useful are polypropylene glycols, such as those described in U.S. Pat. No. 5,643,575. Particular PEG's useful in the methods of the invention are described in Shearwater Polymers, Inc. catalog “Polyethylene Glycol and Derivatives 2001.” The disclosure of each of these references is incorporated herein by reference. As is known to those of skill in the art such polyalkylene oxide polymers can be functionalized at their termini to introduce functional groups, including those listed in Table 1, to react with the conjugate components to be connected.

Although polyalkylene oxides and polyethylene glycols can vary substantially in average molecular weight, preferably, the polyalkylene oxide moieties have an average molecular weight of from about 2,000 to about 136,000 Da in most linkers used. Shorter linkers also are contemplated, for example those that could more specifically be considered oligoethylene glycol linkers, such as those having from 2 to about 15 ethylene glycol units, such as from about 4 to about 12 ethylene glycol units, such as, on average 5, 6, 7, 8, 9, 10 or 11 ethylene glycol units. More preferably, the polyethylene glycol linkers used herein have weight average molecular weight of from about 3,400 to about 65,000 Da, with a weight average molecular weight of from about 3,400 to about 20,000 Da, such as from about 4,000 to about 8,000 Da being most preferred.

As discussed above, particular examples of linker compounds include oligo- or poly ethylene glycol (PEG) chains with reactive terminal groups to form covalent bonds with the two conjugate components to be linked. PEG linkers with groups set forth in Table 1 are commercially available, and/or can be synthesized by those of skill in the art. Another example of a linker molecule is a succinic acid moiety. Succinic acid anhydride was used to prepare linkages between nanocarriers (such as polyglycerol) and therapeutic agents, such as antitumor agents, for example taxanes, including paclitaxel. In certain examples succinate linkers were used in combination with PEG-derived linkers, for example a succinate linker was directly bonded to a PEG-derivative and the succinate/PEG linker was used to link two conjugate components together. In one particular example, such a succinate/PEG linker was used to link polyglycerol to other components, by way of example an imaging agent, such as the near-infrared dye Cy5.5.

The succinate linker is described for exemplary purposes, however it is understood that other alkylene linkers, such as alkylene chains having the formula (CH₂)ₙ, optionally interrupted by one or more heteroatoms, in particular oxygen, and wherein n is an integer from 2 to about 12, such as 3, 4, 5, 6, 7, 8, 9, 10 or 11. As with the succinate linker, such linker groups include a functional group, such as those listed in Table 1 (or an equivalent thereof), for reaction with a nanocarrier, imaging agent, therapeutic agent and/or targeting agent to link one or more of these components to another.

In certain embodiments the disclosed conjugate compounds are used as prodrugs that deliver a therapeutic agent to a target. Thus it may be desirable to introduce labile linkages, e.g. containing spacer elements that are biodegradable or chemically sensitive or which incorporate enzymatic cleavage sites. In general, the presently disclosed conjugates may contain cleavable groups such as vicinal glycol, azo, sulfone, ester, thioester or disulphide groups linking two or more of the targeting, nanocarrier, and therapeutic or imaging components. In one embodiment, such groups are readily biodegraded in the presence of esterases in vivo, but are stable in the absence of such enzymes. Thus, the linkers can include such labile groups.

Linkers can include, for example, ethylene glycol, propylene glycol, ethanolamine, ethylenediamine, oligomers and derivatives thereof. Other representative spacer elements include oligosaccharides and polysaccharides, such as polygalacturonic acid, glycosaminoglycans, heparinoids, cellulose, alginates, chitosans carrageenans, dextran, aminodextran; peptides, polyamino acids and esters thereof, as in homo- and co-polymers of lysine, glutamic acid and aspartic acid; and oligonucleotides. In certain embodiments such linkers may contain enzyme cleavage sites.

Spacer elements may typically consist of aliphatic chains that optionally are interrupted by one or more heteroatoms and effectively separate the reactive moieties of the linker by distances of between about 0.5 and 300 nanometers. In one embodiment the spacer elements include polyethylene glycol derivatives, such as oligoethylene glycol and polyethylene glycol. Such polymeric structures, hereinafter referred to as PEGs, are simple, neutral polyethers which have been given much attention in biotechnical and biomedical applications (Milton Harris, J. (ed) “Poly(ethylene glycol) chemistry, biotechnical and biomedical applications” Plenum Press, New York, 1992). PEGs are soluble in most solvents, including water, and are highly hydrated in aqueous environments, with two or three water molecules bound to each ethylene glycol segment; this hydration phenomenon has the effect of preventing adsorption either of other polymers or of proteins onto PEG-modified surfaces. Furthermore, PEGs may readily be modified and bound to other molecules with only little effect on their chemistry. Their advantageous solubility and biological properties are apparent from the many possible uses of PEGs and copolymers thereof, including block copolymers such as PEG-polyurethanes and PEG-polypropylenes. Appropriate molecular weights for PEG spacers used in the presently disclosed conjugates typically are from about 120 daltons to about 20 kilodaltons.

### V. COMPOSITIONS AND METHODS

Another aspect of the disclosure includes pharmaceutical compositions prepared for administration to a subject and which include a therapeutically effective amount of one or more of the currently disclosed compounds. Disclosed also are methods for administering the disclosed compounds and compositions. The methods may include selecting a subject in need of treatment with the composition, for example by performing a diagnostic test to determine that the subject is in need of the treatment. In some examples, the subject is selected as having a tumor, such as a tumor responsive to a chemotherapeutic agent carried by the nanoparticle, such as a tumor responsive to a taxane such as paclitaxel. The therapeutically effective amount of a disclosed compound will depend on the route of administration, the type of mammal that is the subject and the physical characteristics of the subject being treated. Specific factors that can be taken into account include disease severity and stage, weight, diet and concurrent medications. The relationship of these factors to determining a therapeutically effective amount of the disclosed compounds is understood by those of ordinary skill in the art.

Methods are disclosed herein for treating conditions characterized by abnormal or pathological proliferative activity. Such conditions that can be treated according to the disclosed method include those characterized by abnormal cell growth and/or differentiation, such as cancers and other neoplastic conditions. Typical examples of proliferative disorders that can be treated using the disclosed compounds and compositions include leukemias, including acute leukemias (such as acute lymphocytic leukemia, acute myelocytic leukemia, acute myelogenous leukemia and myeloblastic, promyelocytic, myelomonocytic, monocytic and erythroleukemia), chronic leukemias (such as chronic myelocytic (granulocytic) leukemia, chronic myelogenous leukemia, and chronic lymphocytic leukemia), polycythemia vera, lymphoma, Hodgkin's disease, non-Hodgkin's lymphoma (indolent and high grade forms), multiple myeloma, Waldenstrom's macroglobulinemia, heavy chain disease, myelodysplastic syndrome, hairy cell leukemia, myelodysplasia, sarcomas and carcinomas, include fibrosarcoma, myxosarcoma, liposarcoma, chondrosarcoma, osteogenic sarcoma, and other sarcomas, synovioma, mesothelioma, Ewing's tumor, leiomyosarcoma, rhabdomyosarcoma, colon carcinoma, lymphoid malignancy, pancreatic cancer, breast cancer, lung cancers, ovarian cancer, prostate cancer, hepatocellular carcinoma, squamous cell carcinoma, basal cell carcinoma, adenocarcinoma, sweat gland carcinoma, sebaceous gland carcinoma, papillary carcinoma, papillary adenocarcinomas, medullary carcinoma, bronchogenic carcinoma, renal cell carcinoma, hepatoma, bile duct carcinoma, choriocarcinoma, Wilms' tumor, cervical cancer, testicular tumor, bladder carcinoma, and CNS tumors (such as a glioma, astrocytoma, medulloblastoma, craniopharyogioma, ependymoma, pinealoma, hemangioblastoma, acoustic neuroma, oligodendroglioma, menangioma, melanoma, neuroblastoma and retinoblastoma), and the like.

Methods also are disclosed herein for treating non-cancerous conditions. For example, methods are disclosed herein for improving vascular function in a subject. The methods include administering to the subject a therapeutically effective amount of a compound disclosed herein to improve vascular function. In one embodiment, the subject has atherosclerosis.

The therapeutically effective amount of the compound or compounds administered can vary depending upon the desired effects and the factors noted above. Typically, dosages will be between about 0.01 mg/kg and 250 mg/kg of the subject's body weight, and more typically between about 0.05 mg/kg and 100 mg/kg, such as from about 0.2 to about 80 mg/kg or from about 5 to about 40 mg/kg of the subject's body weight. Thus, unit dosage forms can be formulated based upon the suitable ranges recited above and a subject's body weight. In one embodiment, a therapeutically effective amount is effective to treat a condition associated with cardiovascular dysfunction (for example atherosclerosis). In one such embodiment, a therapeutically effective amount is an amount sufficient to increase blood flow.

The compounds disclosed herein may be administered orally, topically, transdermally, parenterally, via inhalation or spray and may be administered in dosage unit formulations containing conventional non-toxic pharmaceutically acceptable carriers, adjuvants and vehicles.

Typically, oral administration or administration via injection is preferred. The inhibitors may be provided in a single dosage or chronically, dependent upon the particular disease, condition of patient, toxicity of compound and other factors as will be recognized by a person of ordinary skill in the art.

The therapeutically effective amount of the compound or compounds administered can vary depending upon the desired effects and the factors noted above.

Pharmaceutical compositions for administration to a subject can include carriers, thickeners, diluents, buffers, preservatives, surface active agents and the like in addition to the molecule of choice. Pharmaceutical compositions can also include one or more additional active ingredients such as antimicrobial agents, anti-inflammatory agents, anesthetics, and the like. Pharmaceutical formulations can include additional components, such as carriers. The pharmaceutically acceptable carriers useful for these formulations are conventional. Remington's Pharmaceutical Sciences, by E. W. Martin, Mack Publishing Co., Easton, Pa., 19th Edition (1995), describes compositions and formulations suitable for pharmaceutical delivery of the compounds herein disclosed.

In general, the nature of the carrier will depend on the particular mode of administration being employed. For instance, parenteral formulations usually contain injectable fluids that include pharmaceutically and physiologically acceptable fluids such as water, physiological saline, balanced salt solutions, aqueous dextrose, glycerol or the like as a vehicle. For solid compositions (for example, powder, pill, tablet, or capsule forms), conventional non-toxic solid carriers can include, for example, pharmaceutical grades of mannitol, lactose, starch, or magnesium stearate. In addition to biologically-neutral carriers, pharmaceutical compositions to be administered can contain minor amounts of non-toxic auxiliary substances, such as wetting or emulsifying agents, preservatives, and pH buffering agents and the like, for example sodium acetate or sorbitan monolaurate.

It is specifically contemplated in some embodiments that inhibitor delivery is via an injected and/or implanted drug depot, for instance comprising multi-vesicular liposomes such as in DepoFoam (SkyePharma, Inc, San Diego, Calif.) (see, for instance, Chamberlain et al. Arch. Neuro. 1993, 50, 261-264; Katri et al. J. Pharm. Sci. 1998, 87, 1341-1346; Ye et al., J. Control Release 2000, 64, 155-166; and Howell, Cancer J. 2001, 7, 219-227).

A method is provided herein for the in vivo or in vitro detection of particular tissues or cells. An in vivo detection method can localize any target tissue or cell, such as atherosclerotic lesions, neo-vascularized and inflamed tissue areas, or a tumor, selected by choice of an appropriate targeting agent. In one embodiment, a subject having or suspected of having cancer is treated with a conjugate compound including an imaging agent. After a sufficient amount of time for the conjugate to localize to the tumor or cell in the subject, the tumor or cell can be detected. In one specific, non-limiting example detection of a cancer cell is accomplished using a technetium-99m labeled conjugate. Other specific, non-limiting examples of detection include fluorescence imaging.

In one specific embodiment, the detection step is performed prior to surgery. In another embodiment, the detection step is performed during surgery, for example to detect the location of the tumor prior to removing it, as in radioimmunoguided surgery.

In yet another embodiment, the detection step is performed after surgery to ensure the complete removal of the tumor, or to detect a recurrence of the tumor. In one specific, non-limiting example, a radiolabeled immune complex is detected using a hand-held gamma detection probe.

The in vitro detection method can be used to screen any biological sample containing any tumor or cell that expresses a targeted group as discussed below. Such samples include, but are not limited to, tissue from biopsies, autopsies, and pathology specimens. Biological samples also include sections of tissues, such as frozen sections taken for histological purposes. Biological samples further include body fluids, such as blood, serum, saliva, or urine. A biological sample is typically obtained from a mammal, such as a human subject. In one embodiment the subject has a condition comprising breast cancer, bladder cancer, bone cancer, cervical cancer, colon cancer, central nervous system cancer, esophageal cancer, gall bladder cancer, gastrointestinal cancer, head and neck cancer, laryngeal cancer, leukemia, lung cancer, melanoma, ovarian cancer, prostate cancer or renal cancer.

### EXAMPLES

The foregoing disclosure is further explained by the following non-limiting examples.

### Example 1

### Synthesis of GT/GFT Conjugate Compounds

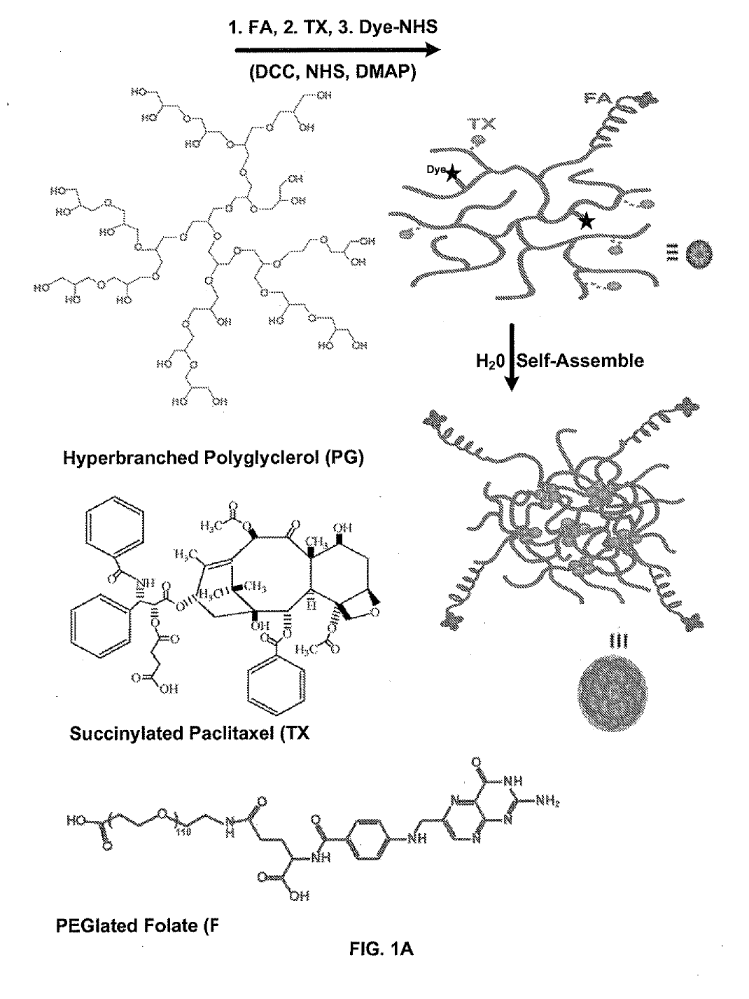

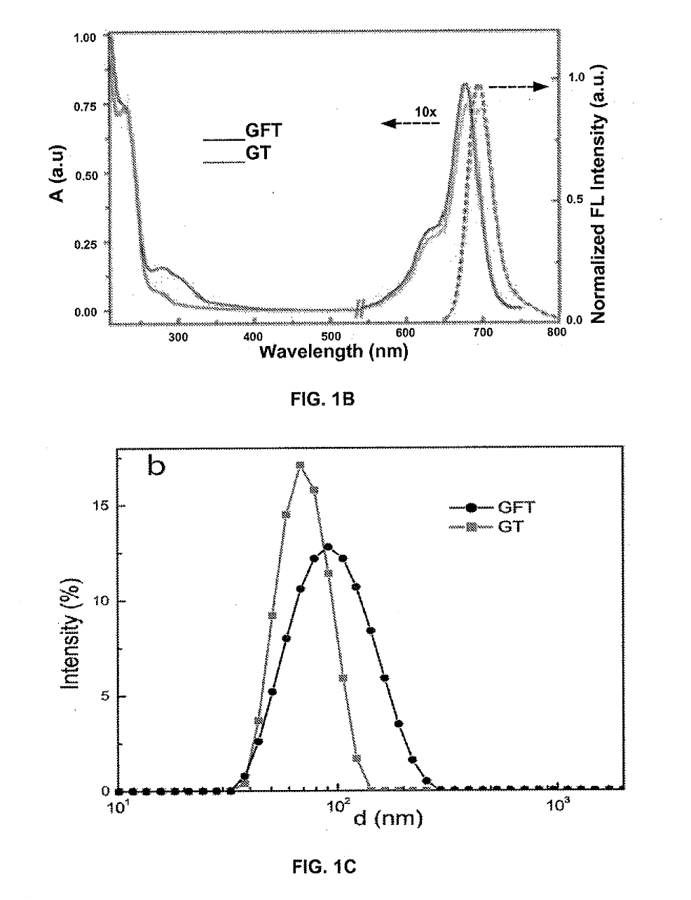

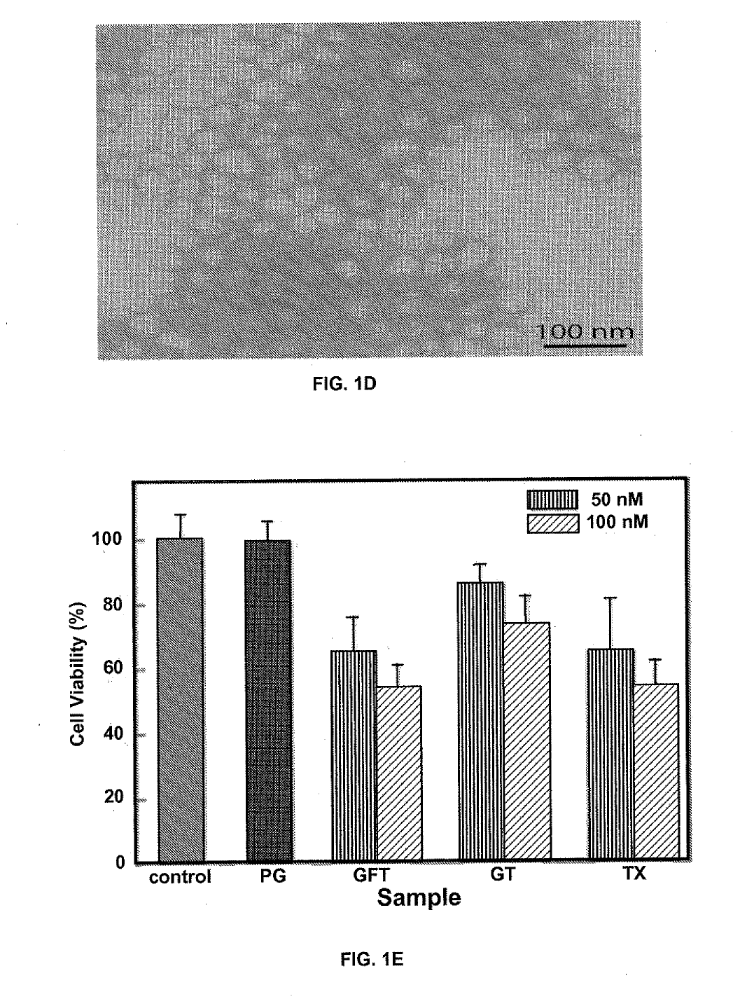

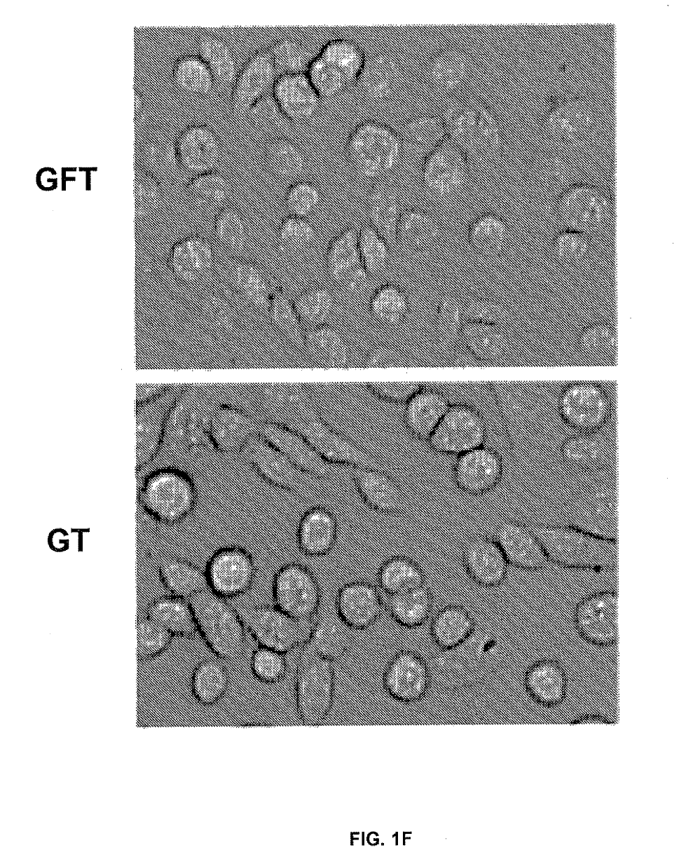

FIG. 1A shows the design and conjugation chemistry of a multi-component polymer-drug conjugate for targeted cancer imaging and therapy, using polyglycerol (PG, molecular weight=20,000 Da) as a hosting carrier for the other functional units, paclitaxel (TX) as an anticancer therapeutic agent, folate (FA) with a polyethylene glycol (PEG, molecular weight=5000 Da) linker as a targeting ligand and the near infrared (NIR) fluorescent dye cy55 as an imaging tag for tracking the delivery of the conjugate in-vitro and in-vivo. The controlled synthesis of PG was achieved via ring opening multibranching polymerization of glycidol under slow monomer addition conditions. TX was linked to the PG backbone through a degradable linker (succinic acid). The folate receptor was chosen as a targeting agent because it is highly overexpressed in many types of human cancer, such as head-neck and breast cancer, due to the increasing need of nutrients for cancer cells to support their propagation. The cy55 dye, having an emission maximum at about 700 nm, allows the delivery of the PG-drug conjugates to be non-invasively imaged. Its wavelength range of 650-900 nm provides an excellent imaging window for in vivo optical imaging due to the limited absorption of blood and water in this wavelength range. The coupling of these functional components to PG carriers was facilitated by the mild reaction catalyzed by N,N′-Dicyclohexylcarbodiimide (DCC) and N-hydroxysuccinimide ester (NHS). The composition of the conjugates can be well controlled by changing their feeding ratios.

PG was synthesized as reported previously. Additional details of preparations of succinylated paclitaxel derivative (TX), peglated folate (FA) and FA-functionalized PG are available in Examples 9 and 10. In summary, conjugation of PG with TX was catalyzed by DCC/NHS/DMAP. Briefly, 200 mg PG (MW=20,000 Da) was dissolved in 10 mL of anhydrous N,N′-dimethylformamide (DMF); a solution of 30 mg succinyl-paclitaxel in 1 mL of DMF was added, and the solution was cooled in an ice-water bath. A solution of 9 mg DCC and 9 mg N-hydroxysuccinimide (NHS) in 0.5 mL of DMF was added upon stirring followed by a solution of 1 mg of 4-dimethylamino pyridine (DMAP) in 0.5 mL of DMF. The reaction mixture was kept in the ice-water bath for 5 min and then 18 h at room temperature. The precipitated dicyclohexylcarbaminde was filtered off and the solution was precipitated with acetone. After repeating this cycle three times the residue was dried under vacuum, dissolved in distilled water and dialyzed (MW cutoff 10,000Da) for 2 days against water. After lyophilization the conjugate was obtained. For targeted conjugates with FA, FA-functionalized PG instead of PG was used in the reaction, and the reaction and purification process are the same as above.

The cy5.5 labeled polymer drugs were prepared by reacting the conjugates with NHS-activated dye, and the free unattached dye was removed by extensive dialysis (Cutoff=10,000Da) followed by three time centrifuging filtration through the Centricon devices (Cutoff—10,000Da).

### Example 2

### Nanoparticle Characterization

UV-vis spectra of the nanoparticles of Example 1 were obtained on a Shimadzu (UV-2401) spectrometer using quartz cuvettes. Fluorescent spectra were recorded using a PTI fluorometer with excitation wavelength of 625 nm for cy5.5. Transmission electron microscopy (TEM) observation was performed on a Hitachi H7500 electron microscopy at an acceleration voltage of 75 kV. Dynamic light scattering and zeta potential measurement were carried out on a Malvern Zetasizer Nano ZS90 at 25° C., and the results are the average value of three consecutive measurement.

Poor water-solubility of TX has been a significant hindrance to its clinical use. After being conjugated to the PG carrier, the water solubility of TX was greatly enhanced, and 10 mg/mL TX equivalent solution of the conjugate can be readily prepared. In UV-vis spectra (FIG. 1B) of these conjugates, absorption peaks of TX at 230 nm and FA at 280 nm are clearly visible; the conjugation of cy55 to PG did not affect their fluorescent properties including emission wavelength and quantum yield.

### Example 3

### Single Particle Imaging and Disassembly of Nanoparticles in Serum

Dye-labeled nanoparticles were dissolved in fetal bovine serum with a dye concentration of 100 nM, and the solution was incubated at 37° C. At a predetermined time interval, 2 μl of the solution was added on a glass slide and spread with a cover slip. Photographic images were taken on an Olympus fluorescent imaging microscopy with 300 ms exposure time.

Dynamic light scattering (DLS) and transmission electron microscope (TEM) (FIG. 1C) revealed that the conjugates can self-assemble into uniform nanoparticles in water, with a hydrodynamic size of 70-100 nm (measured by DLS) and an average size at the dried state about 50 nm measured by TEM (FIG. 1D). The FA-targeted nanoparticle (GFT) is 10-15 nm larger than the non-targeted one (GT), which is due to an addition layer of PEG spacer added by the FA ligands. This self-assembly behavior is driven by the hydrophobic interactions between TX linked on the polymer carrier, because the nanoparticle disappeared after adding a solvent of TX such as DMF. It is believed that the nanoparticles formed by the conjugates have a structure resembling hydrogel nanoparticles (FIG. 1A), however, instead of being chemically crosslinked, these nanoparticles are physically crosslinked in an aqueous environment (such as the blood) by the hydrophobic domains formed by TX molecules. Based on hydrodynamic sizes of PG (about 4.5 nm for 20,000 Da polymers) determined by DLS and geometric calculations (assuming a maximum packing density of 0.64 for randomly closed-packed configuration), it is estimated that one 80-nm self-assembled nanoparticle consists of about 3600 individual PG-drug conjugates. Hence in certain disclosed embodiments, each self-assembled nanoparticle contains 3000-4000 individual PG-drug conjugates, for example 3200-3800, such as 3400-3700 conjugates. In certain examples, the size of the self-assembled nanoparticle is 70-90 nm in diameter.

### Example 4

### Quantification of Release Profile of Conjugated TX

Polymer drug conjugates (20 mg) were dissolved in 2 mL PBS and sealed in a dialysis bag (molecular cutoff 3500). The dialysis bag was incubated in 25 mL PBS at 37° C., and 100 μL aliquots were taken out at predetermined time intervals. Released TX was quantified with UV-vis spectrometer by using a calibration curve from 2 μg to 50 μg TX equivalent.

### Example 5

### Analysis of Cytotoxicity and Cellular Uptake

The cytotoxicity of these conjugates was tested on multiple cancer cell lines. Cytotoxicity effects of the PG-drug conjugates were evaluated by using a standard 3-(4,5-dimethylthiazol-2-yl)-2,5-diphenyl tetrazolium bromide (MTT) assay. The cells were cultured in RPMI 1640 containing 100 units/mL penicillin G, 100 ug/mL streptomycin sulfate. The medium was supplemented with 10% heat-inactivated fetal bovine serum. The tumor cells were cultured in tissue culture flask in a humidified incubator at 37° C. in an atmosphere of 5% CO2 and 95% air. Cancer cells (about 60% confluency) were treated with drug conjugates or free drug (added as DMSO solution) for 4 hours, and then culture medium was replaced with fresh one. After 48 hours incubation, MTT solution was added (incubated for 2 hours) and UV-vis absorption was measured at 490 nm with 670 nm as the reference wavelength.

To image the cellular uptake of the polymer drug, the dye labeled nanoparticles (100 ng TX equivalent) incubated with cells for 2 hours, and then culture medium containing drugs were removed, washed three times with PBS and finally replaced with fresh medium. Cell imaging was obtained on Olympus fluorescent imaging microscopy equipped with a Nuance spectral imaging system (500-900 nm).

The in-vitro delivery of these nanoparticles was evaluated on a breast cancer cell line, MDA-MB-231, which shows high expression levels of folate receptor. FIG. 1E summarizes the cytotoxicity test results for the conjugates compared to free TX added as DMSO solution. At two different concentrations, GFT nanoparticles showed higher toxicity than GT conjugates, indicating the increasing uptake of the targeted nanoparticles through the receptor-mediated endocytosis. This result also was corroborated by the significantly higher cy5.5 fluorescence of targeted nanoparticles in the cells (FIG. 1F) when the dye-labeled nanoparticles were used. The fact that the conjugate without targeting ligands also killed a significant amount of cells may be due to the drug molecules being released during the 4 hour incubation, or a very small number of nanoparticles taken up by the cells non-specifically. We also noted that PG did not show any capability to kill the cells, confirming the biocompatibility of this polymer carrier.

### Example 6

### Animal Models

Athymic nude mice were purchased from Charles River and housed in a specific pathogen-free animal facility at Emory University. In-vivo imaging, blood circulation and biodistribution studies were performed. MDA-MB-231 tumor model was used for testing the in-vivo imaging of these polymer-drug conjugates. Three weeks before the test, the cells were harvested and 5×10⁶ cells were suspended in a mixture of 50 μL culture medium and 50 μL Matrigel. The cells were injected into the mammary fat pad of athymic nude mice. When the size of the tumor reached 400-500 mm³, the near infrared dye (Cy5.5, GE healthcare) labeled conjugates were systemically injected. Polymer drugs containing 10 nmol of cy5.5 were injected into mice through the tail vein. For in-vivo imaging, mice were anesthetized with an intraperitoneal mixture of ketamine (120 mg/Kg) and xylazine (8 mg/Kg) and imaged with Kodak small animal imaging system with excitation at 625 nm and emission at 700 nm. For biodistribution studies, mice were killed at time point of 6 and 144 hours to collect major organs and tumors for quantification of cy5.5 fluorescence in organs and tumors.

Circulation studies were conducted on healthy animals. After the nanoparticles were injected in the tail vein, 10 μA of blood were withdrawn from the tail vein at predetermined time intervals and diluted to 200 μl for fluorescence measurements. EDTA was used as anticoagulant. Each group consisted of 3 mice.

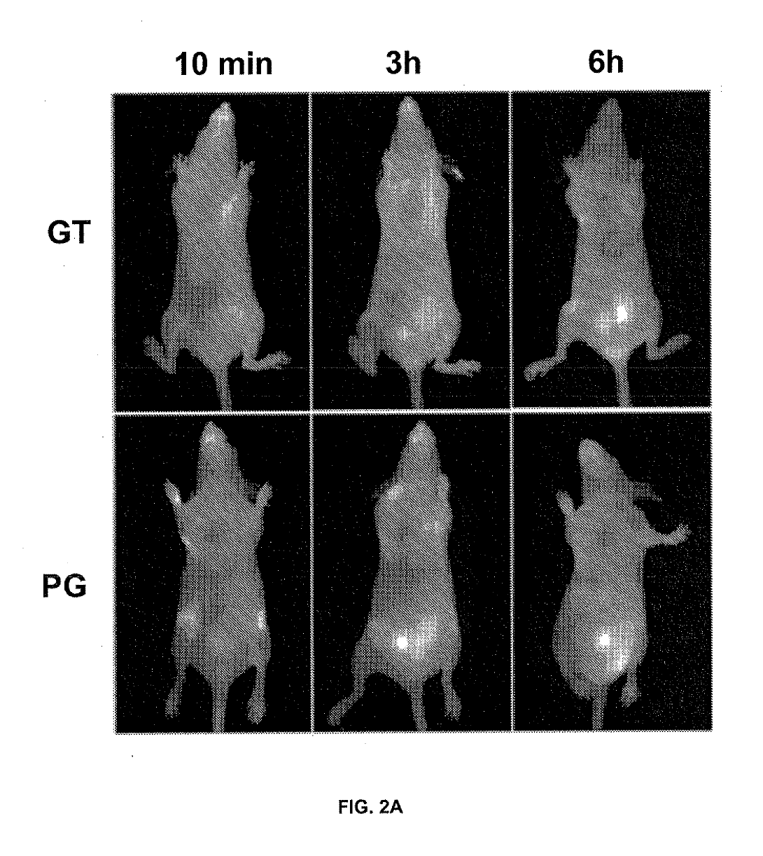

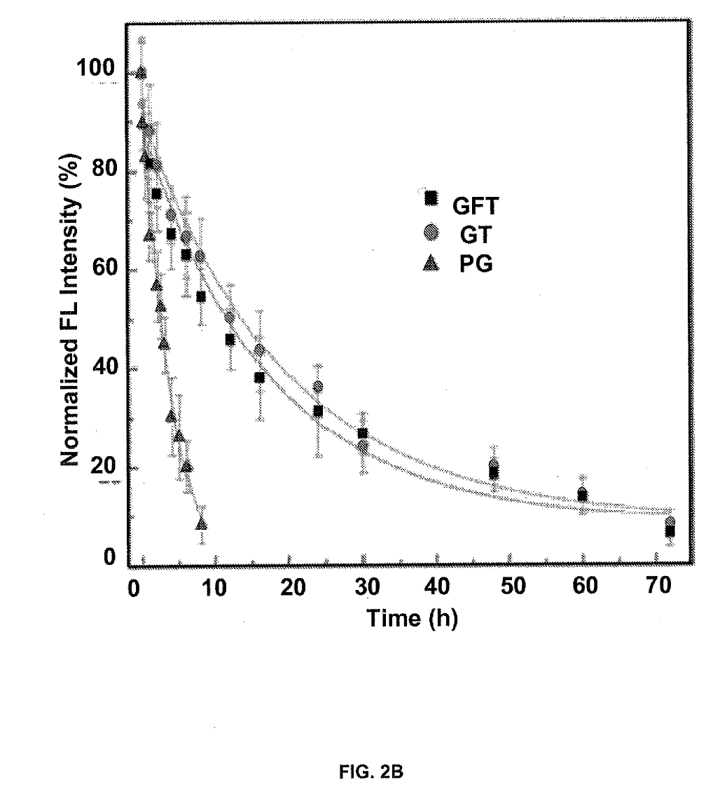

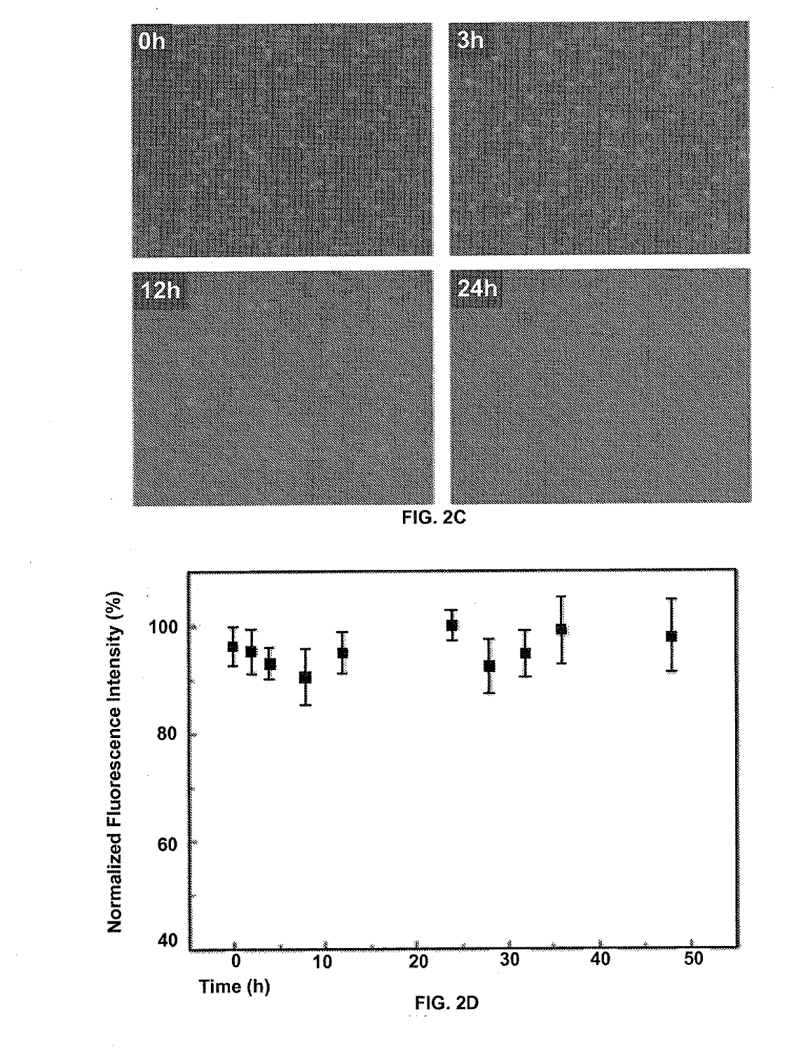

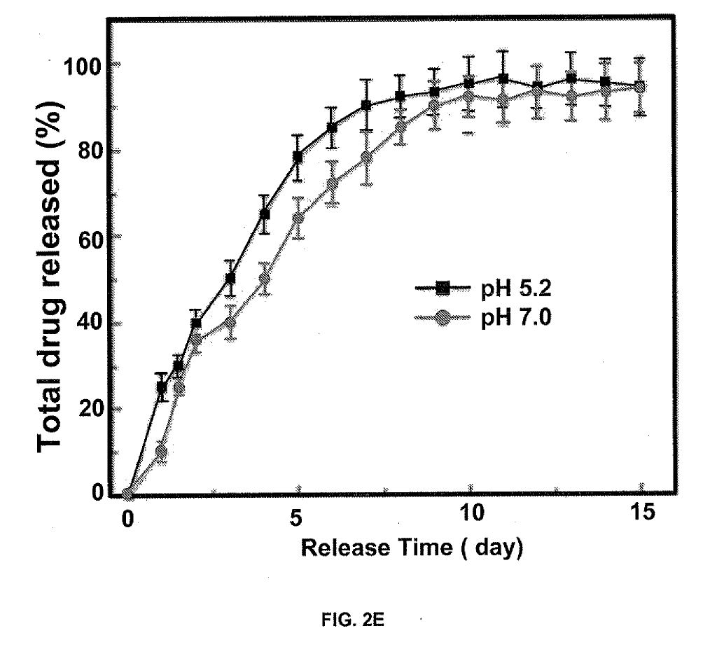

The NIR fluorescent tag in the self-assembled nanoparticles makes it possible to track their in-vivo delivery and blood circulation in mice. FIG. 2A showed that systemic delivery of nanoparticles led to clearly visible blood vessels in mice. Blood circulation of the cy5.5 labeled nanoparticles and PG carrier were followed by measuring the fluorescence of blood samples after injection into the tail vein of healthy mice. The results demonstrate that self-assembled nanoparticles have a longer plasma presence (half-life of 10-12 hours) than their polymer precursors (half life of 3-4 hours) (FIG. 2B). Ten minutes after injection, the fluorescence of PG precursor was already detected in bladder, and the fluorescence in the blood vessels also quickly faded within 3 hours. In contrast, in case of the nanoparticles, these blood vessels remained visible at least 8 hours after injection, and at 10 minutes, fluorescence was not detectable in the bladder. After 3 hours only a weak signal was observed in the bladder where the signal intensity increased over time. The fluorescence spectra detected in the urine of mice is identical to the original spectra, suggesting that it is reliable to use optical imaging to follow the blood circulation. The results showed that nanoparticles with 10% (w:w) of TX and 1-2% (w:w) of FA led to the optimal blood circulation, providing a means for the pretreatment optimization of the conjugate. However, the disclosed particles include a broader range of active agent and targeting agent, such as 8-12% of the active agent (such as a taxane) and 1-5% of a targeting agent (such as FA).

High sensitivity fluorescence imaging (FIG. 2C) found single dye-tagged nanoparticles after the diluted nanoparticle solution was spread on a glass surface. With the same optical setup, individual polymers are not visible because of their exceedingly weak fluorescence signal (0.01% of that of nanoparticles). The several orders of difference in fluorescence intensity offered an optical approach to monitor the process of disassembly of these self-assembled nanoparticles. FIG. 2C showed the decease in the number of fluorescent nanoparticles accompanied by the increase of background signal over a 24 hour incubation in 100% fetal bovine serum. Meanwhile, the overall fluorescence of the solutions measured by a fluorescence spectrometer was relatively unchanged during the time of incubation (FIG. 2D), indicating that the disappearance of fluorescent nanoparticle is associated with disassembly of nanoparticles into small pieces. The slow renal clearance of self-assembled nanoparticles observed from in vivo imaging suggests this disassembly should also occur during the blood circulation because sizes of the nanoparticles are well above the threshold (5-6 nm) of renal clearance.

The self-assembly of nanoparticles is driven by the hydrophobic interactions of hydrophobic molecules (such as the taxanes, for example TX); therefore, the disassembly process is believed to be the result of competition binding of plasma proteins such as albumins to hydrophobic components or partial release of the hydrophobic molecule from the polymer carrier. Incubation of the conjugate in PBS of different pH at 37° C. showed that it took 5 days to release about 80% of the TX at pH 5.2, and the release profile exhibited a pH-dependence (FIG. 2E), with neutral pH slower than the acid condition. The slow release of drug molecules indicates the plasma protein competition leads to the disassembly.

### Example 7

### Therapeutic Efficacy Test

The MDA-MB-231 tumor model was used for testing the in-vivo therapeutic efficacy of these polymer-drug conjugates. MDA-MB-231 tumors were developed in the mammary fat pad of athymic nude mice as discussed above. When the average sizes of the tumor reached 100-120 or ˜30 mm³, mice were randomly sorted into 4 groups (6 mice each group). A paclitaxel equivalent of 30 mg/kg of free drug and polymer drug conjugates were administered i.v. and the injections were performed weekly. Tumor sizes were measured in two dimensions with calipers and volume was calculated using the following formula: Tumor volume=(length×width²)/2. The end-point size of this tumor model was set at 1,500 mm³, 20% weight loss or severe ulceration (more than 20% of tumor areas).

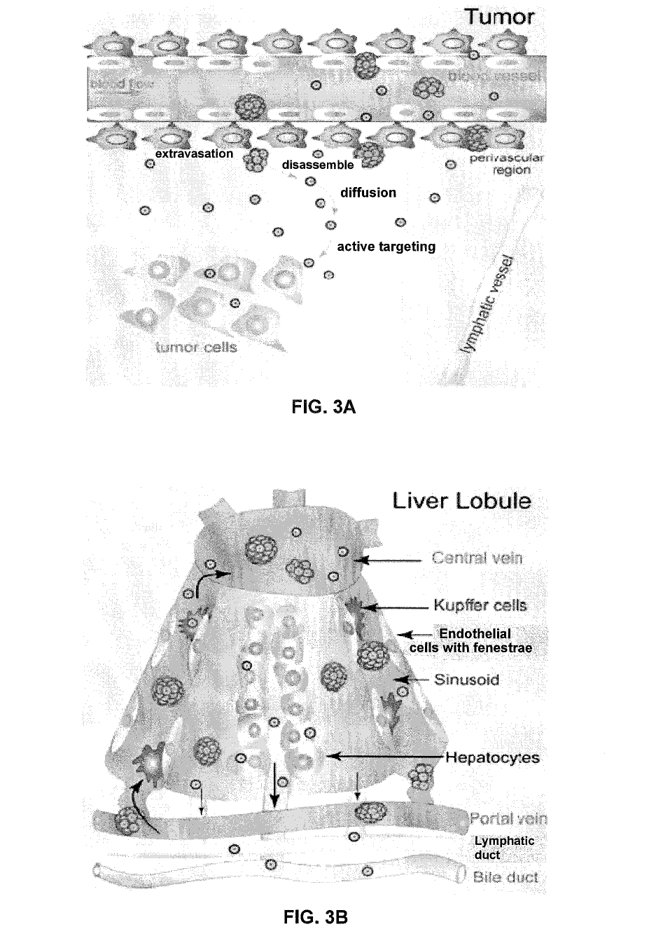

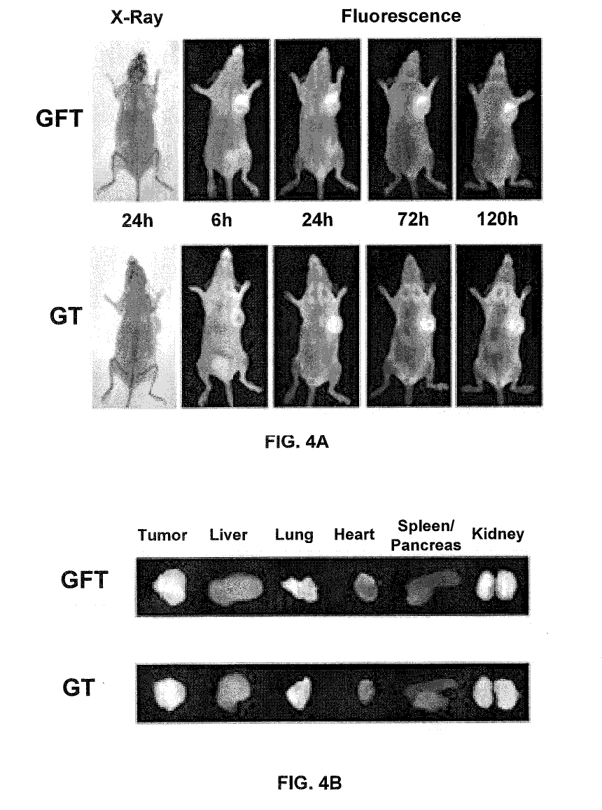

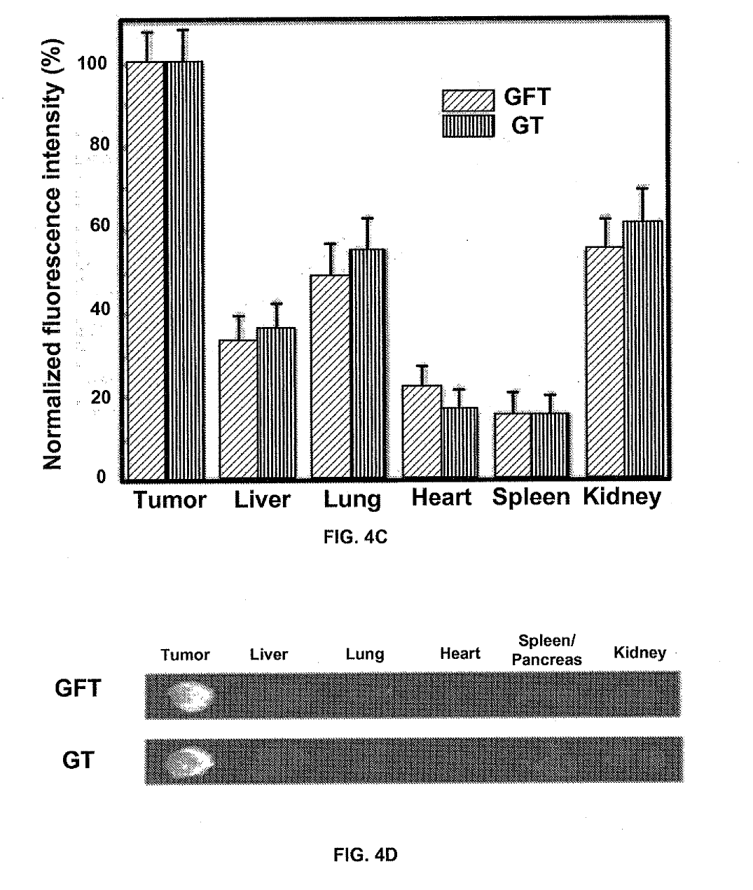

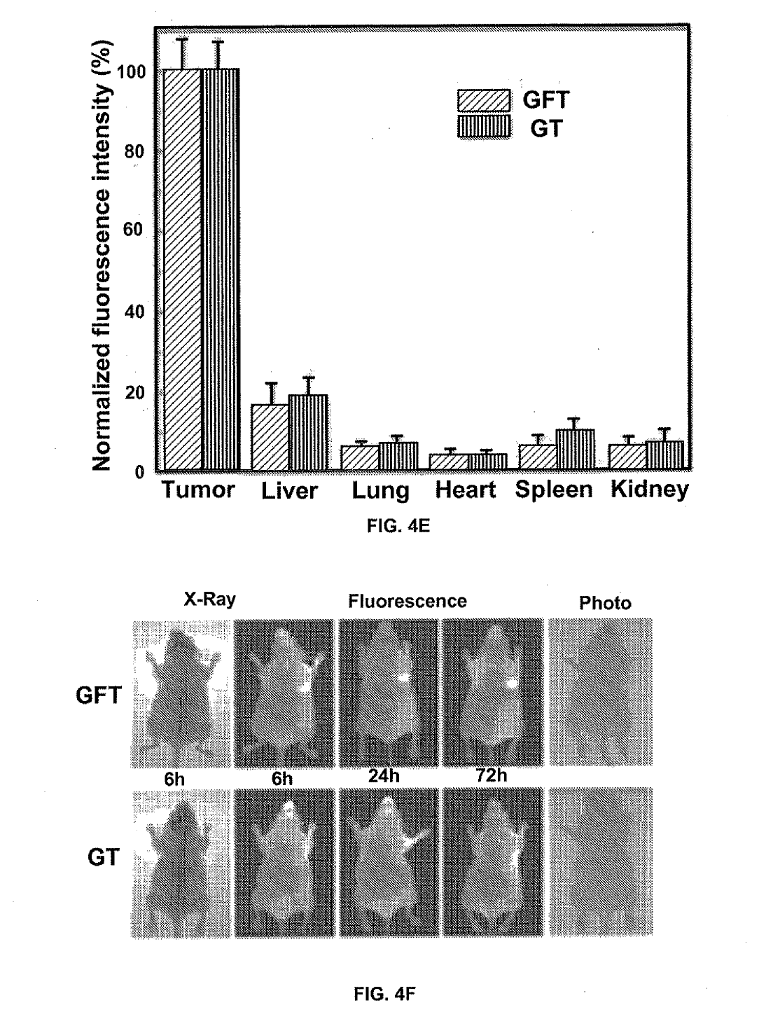

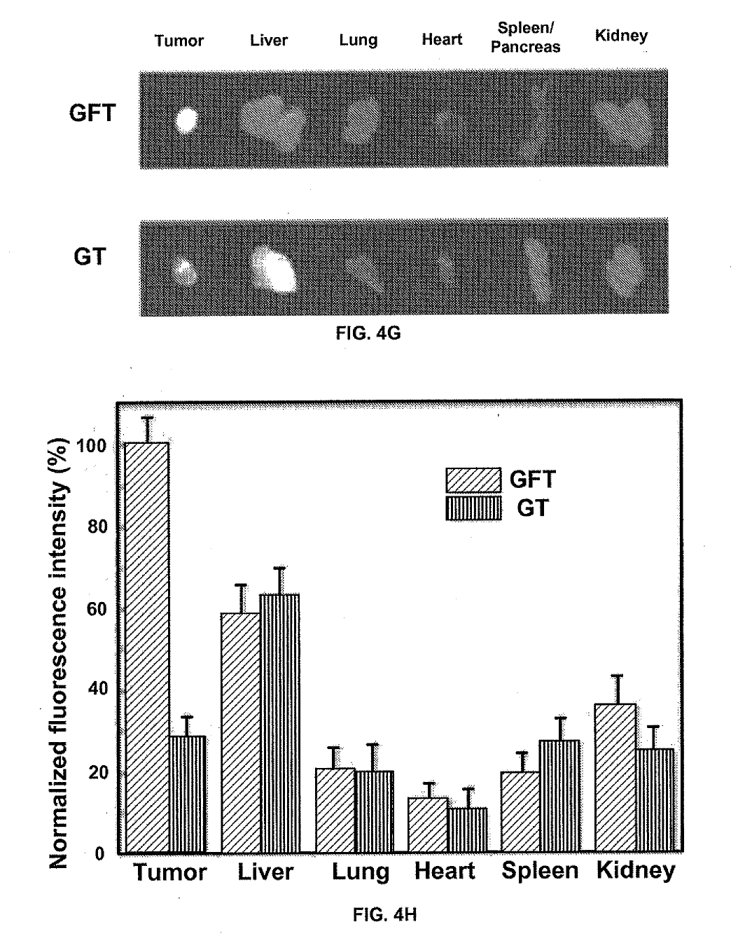

In-vivo delivery of nanoparticles to solid tumors is a complex process, and can be achieved by both passive and active targeting mechanisms, as shown in FIG. 3A. To investigate in-vivo tumor targeting of the self-assembled nanoparticles, nanoparticles both with and without FA ligands were administered systemically (through the tail vein) into the nude mice bearing the human breast tumor (MDA-MB-231) grown orthotopically in the mammary fat pads. The tumors had an average size of 400-500 mm³, and can be clearly visualized through X-ray imaging. In in-vivo optical imaging of the GT and GFT nanoparticles showed accumulation at the tumor sites 6 hours after the injection with the signal intensity continuously increasing over 48 hours and leveling off thereafter. Monitoring up to one week showed the persistent retention of nanoparticles in the tumor (FIG. 4A). The targeting delivery of the nanoparticles with or without FA did not have an obvious difference, consistent with the expectation that targeted and non-targeted nanoparticles exhibit similar biodistribution and tumor localization. This finding also confirms that in this case the amount of nanoparticles delivered to tumor tissues is primarily determined by the extravasation of nanoparticles (FIG. 3A) though the leaky vascular structures formed during the fast and defective angiogenesis. However, the lack of functional lymphatic vessels prevents the drainage of nanoparticles from the tumor mass and led to the persistent tumor accumulation.

FIGS. 4B-E shows the comparative organ distribution of these nanoparticles after a single tail-vein injection. The biodistribution of these nanoparticles from mice sacrificed 6 and 120 hours after injection with the nanoparticles also confirmed the preferential accumulation of nanoparticles in tumors. At 6 hours, the fluorescence of kidney should come from the portion of PG carriers excreted through renal clearance, as shown above; the relatively strong signal in lung is believed to result from the large amount of conjugates retained in the blood pool, as shown in blood circulation results. The consistent information at these two time points is the low uptake of nanoparticles in liver and spleen. This remarkably reduced uptake by the RES is partly due to the stealth effect of PG carriers. However, the dynamic structure of the self-assembled nanoparticles played a role through the disassembly of nanoparticles into individual PG-drug conjugates in blood and tissues, because individual soluble polymers typically show low RES uptake. In contrast, previous reports by many groups showed that PEGylated nanoparticles which stayed intact during the circulation eventually accumulated in liver or spleen with more than 30% of the injected dosage. Gref, R. et al. Biodegradable Long-Circulating Polymeric Nanospheres. Science 263, 1600-1603 (1994).

The disassembly of the nanoparticles also helps enhance the clearance of nanoparticles taken up by the non-specific tissues. For example, as shown in FIG. 3B nanoparticles can exit liver through either vascular approaches (blood vessels and lymphatic systems) or an hepatic pathway (through bile ducts). With the presently disclosed conjugates, the signal in liver at 120 hours exhibited a 50% drop compared with that at 6 hours. Given the fact that the hepatic pathway into bile and into feces often takes a long period of time (several months), it is believed that the clearance of these nanoparticles takes the vascular approach.

The advantages offered by the targeted nanoparticles for imaging tumors of about 400 mm³ in sizes was not as great as the advantages achieved by the FA-targeting for barely palpable tumors (about 2-3 mm in diameter). As shown in FIGS. 4F-G, selective accumulation of FA-targeted nanoparticles could be detected in these very small tumors while non-targeted nanoparticles only showed very little signal above the background. It is known that the angiogenesis process starts after a tumor reaches a certain critical size (about 1 mm). The small tumors in this study should have developed vascular structures. However, these small tumors still have much larger surface area to volume ratio than the larger tumors. Therefore, in small tumors and micrometastasis, while the binding and endocytosis of FA-targeted nanoparticles could lead to their retention in tumor cells, the non-targeted nanoparticles had less uptake by tumor cells.

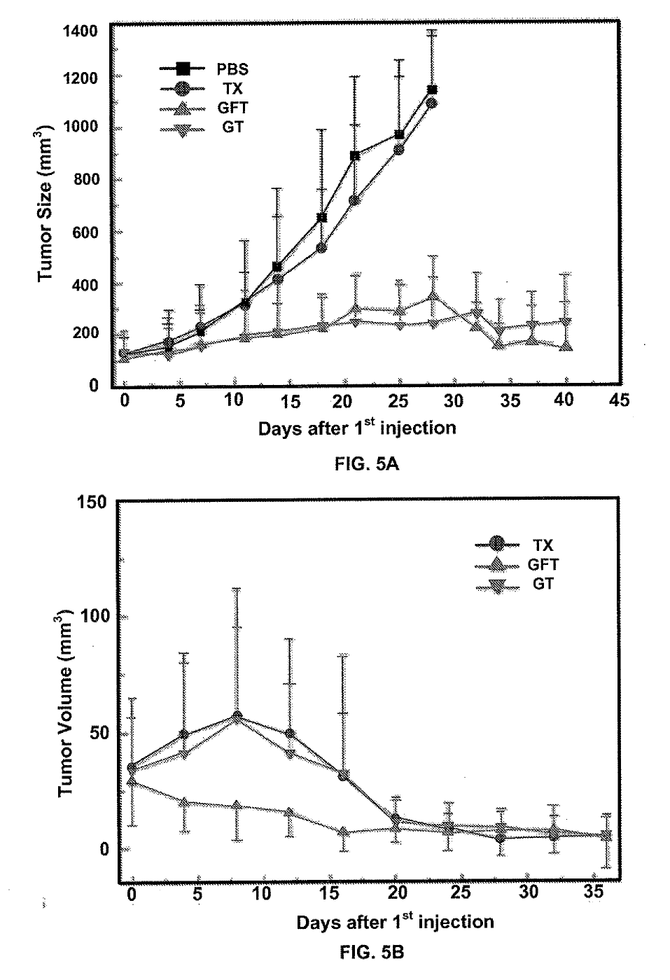

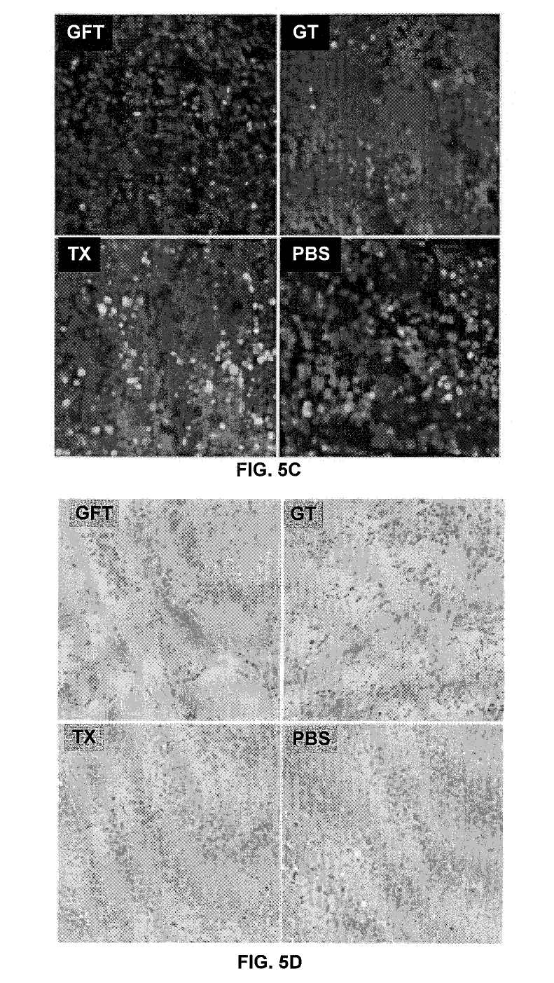

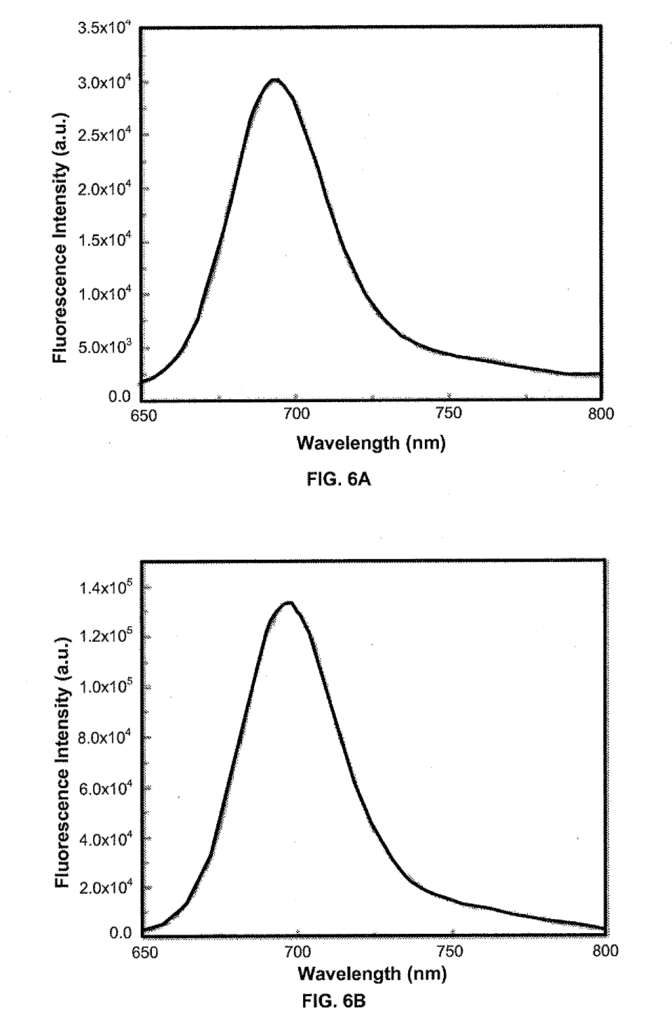

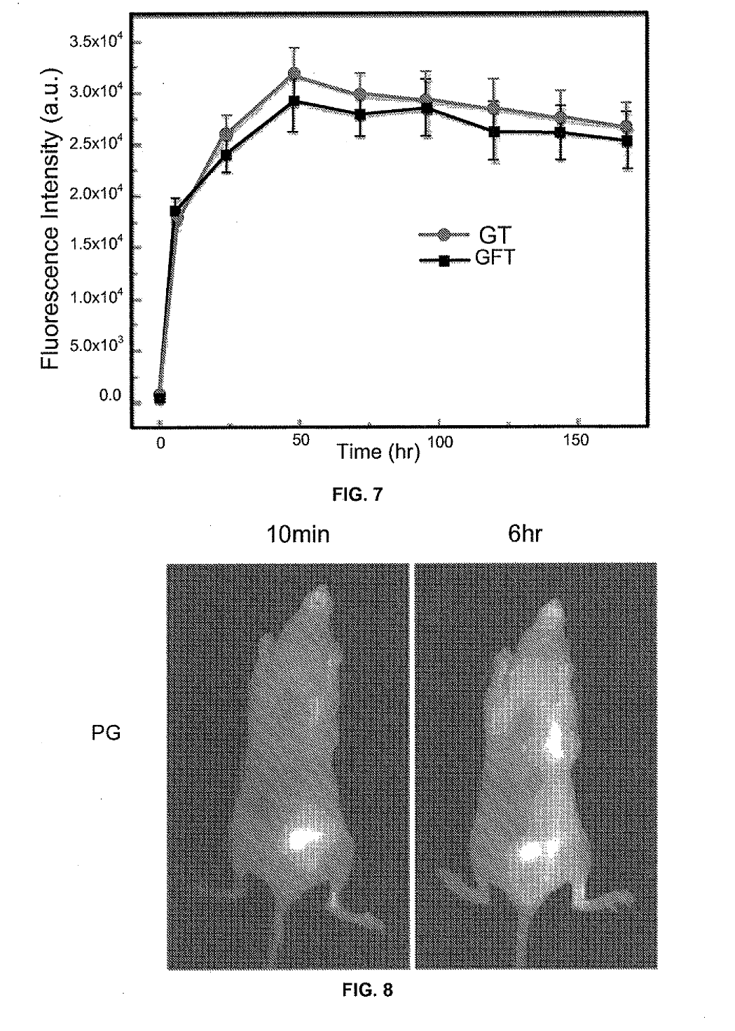

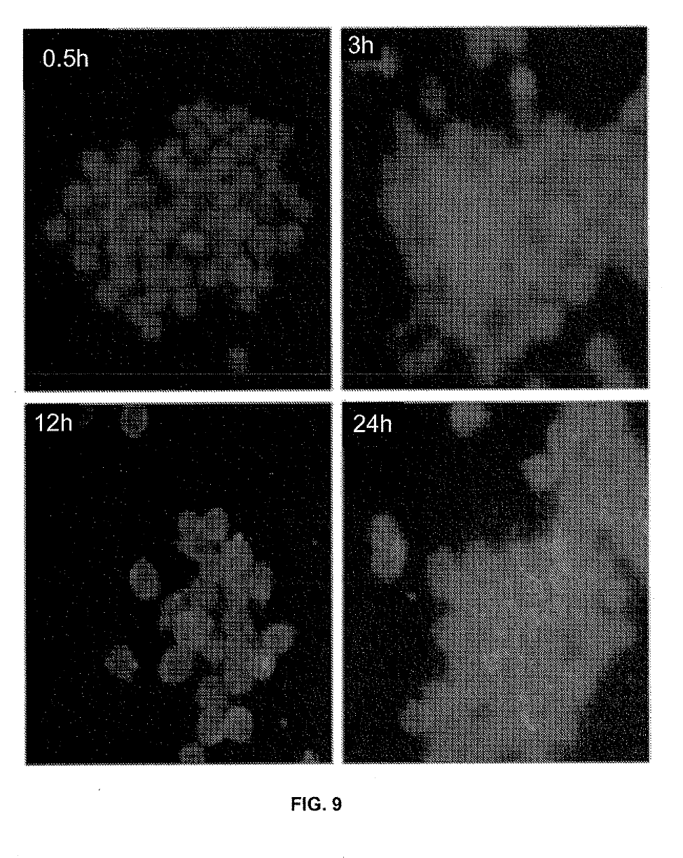

Following the in-vivo blood circulation and tumor targeting tests, the anti-tumor activity of these nanoparticles was evaluated in comparison with free TX using the MDA-MB-231 orthotopic tumor model with two serials trials of tumor sizes of about 120 and 30 mm³. The i.v administration of GFT, GT and free TX was performed for 4 weekly injections at the TX equivalent dose of 30 mg/Kg. As shown in FIG. 5A, both GT and GFT nanoparticles had significant antitumor effects in large tumors, whereas the free drug showed little effect on tumor inhibition, consistent with the in-vivo targeting results. Although GFT had better cellular uptake in the cellar studies (FIG. 1E), the drug molecules released extracellularly also can be absorbed by the tumor cells to a sufficient extent to kill the tumor cells at this dosage. Histological analysis (FIGS. 5C and 5D) of tumor sections from different groups also showed that the injection of nanoparticle drugs induced a significant drop in the expression level of Ki67, a cell proliferation biomarker; and H&E staining showed apoptosis of tumor cells and presence of fibrotic tissues. In contrast, the free drug led to little difference compared to the control (untreated) group.

The therapeutic outcome was also correlated with the imaging results in small-tumor groups. FIG. 5B shows that the targeted nanoparticles quickly induced tumor regression after the first injection whereas the non-targeted nanoparticles and free drug showed a delayed response to the treatment. The leaky tumor vasculature-induced EPR effect would play little role in targeting micro-metastatic lesions. Hence targeted nanoparticles clearly provide advantages in the diagnosis and treatment of these small tumor lesions. Therefore, in some embodiments of the methods, a subject is chosen who has a small tumor, or micro-metastatic disease, and the targeted drug conjugates are administered in an effective amount to the subject to detect or treat the micro-mestatic lesions. In particular examples, the micro-metastatic lesions have tumor sizes (determined as described herein) of less than 100 mm³ for example less than 50 mm³, such as less than 30 mm³.

### Example 8

### Formulation of Nanoparticles by Encapsulation

This example describes formulating nanoparticles using the conjugate described in Example 1 and free therapeutic or imaging agent In this particular example the agent is the taxane paclitaxel. Briefly, 100 mg of the conjugate is prepared and 20 mg free paclitaxel is dissolved in 3 mL DMSO to form a clear solution after 30 seconds sonication. The mixture is put in 20 mL 0.1 M NaHCO₃ solution for 1 hour and dialyzed to remove DMSO (using a MW 3,000 cutoff membrane). The solution is filtered by a 0.2 μM membrane, concentrated by an Ultra-Centrifugal Filter (5,000 MW cutoff) and stored at −20° C. for future usage. The concentration of paclitaxel in the stock solution is detected by UV spectra by diluting it 500-fold.

### Example 9

### Synthesis of Branched Polyglycerol Conjugates

This example describes the synthesis of branched polyglycerol conjugate molecules that include a branched organic polymer and a therapeutic agent and a targeting agent conjugated to the branched organic polymer.

Hyperbranched polyglycerol was prepared according to the protocol of Frey and coworkers (Macromolecules 1999, 32, 4240-4246). A 2′-O— succinyl-paclitaxel derivative (TX) was prepared using 50 mg paclitaxel, 90 mg succinic anhydride were dissolved in 1.2 mL of pyridine for 3 hours at room temperature and the progress of reaction was followed by TLC (acetone/chloroform, 1:1, v/v). Then the solvent was evaporated under vacuum to dryness. The residue was treated with 2 mL of distilled water, stirred for 30 minutes and filtered. The residue was dissolved in acetone followed the slow addition of distilled water. The resultant precipitate was collected by filtration giving a yield was 95%.

Synthesis of PEGlated folate (FA) was prepared by dissolving folic acid (50 mg) in anhydrous dimethyl sulfoxide (DMSO, 15 mL). The dissolved folic acid was activated by dicyclohexyl carbodiimide (DCC) and N-hydroxy succinimide (NHS) (molar ratio, folate:DCC:NHS) 1:1.2:2.4) at room temperature under nitrogen for 30 minutes. The bifunctional PEG derivative (COOH-PEG-NH2, 500 mg), dissolved in 2 mL of DMSO was added to the activated folate solution. The folate conjugation was performed under nitrogen at room temperature for 2 hours. The resultant solution was filtered through 0.45 μm filter unit, dialyzed against deionized water (MWCO 1,000), and lyophilized.

The polyglycerol polyethylene glycol-linked folate conjugate [PG-PEGlated folate (PG-FA)] was prepared by adding a solution of 50 mg PEGlated folate in 2 mL of DMF to 200 mg PG (Mw=20,000 Da) dissolved in 6 mL of anhydrous N,N′-dimethylformamide (DMF); the resultant solution was cooled in an ice-water bath. A solution of 3 mg DCC and 3 mg N-hydroxysuccinimide (NHS) in 1.2 mL of DMF was added with stirring, followed by a solution of 1 mg of 4-DMAP in 0.5 mL of DMF. The reaction mixture was kept in the ice-water bath for 20 minutes and then 18 hours at room temperature. The precipitated dicyclohexyl urea was filtered off and the solution was precipitated with acetone and filtered. After repeating the precipitation/filtration cycle three times, the solution was evaporated under vacuum. Yield: 200 mg.

A polyglycerol-paclitaxel conjugate (PG-2′-O— succinyl-paclitaxel; conjugate GT) was prepared as set forth. Two hundred of polyglycerol (PG, MW=20,000 Da) was dissolved in 10 mL of anhydrous N,N′-dimethylformamide (DMF); a solution of 30 mg succinyl-paclitaxel in 1 mL of DMF was added, and the solution was cooled in an ice-water bath. A solution of 9 mg DCC and 9 mg N-hydroxysuccinimide (NHS) in 0.5 mL of DMF was added upon stirring followed by a solution of 1 mg of 4-DMAP in 0.5 mL of DMF. The reaction mixture was kept in the ice-water bath for 5 minutes and then 18 hours at room temperature. The precipitated dicyclohexyl urea was filtered off and the solution was precipitated with acetone. After repeating this precipitation/filtration cycle three times the residue was dried under vacuum, dissolved in distilled water and dialyzed for 2 days against water. After lyophilization the conjugate was obtained. Yield: 190 mg. The conjugation of paclitaxel was confirmed by NMR and UV spectroscopy. The amount of paclitaxel linked to the conjugate was 12 wt. % as determined by UV spectroscopy.

To assemble a polyglycerol, folate, paclitaxel conjugate 200 mg PG-FA (MW=20,000 Da) was dissolved in 10 mL of anhydrous N,N′-dimethylformamide (DMF); a solution of 30 mg succinyl-paclitaxel in 1 mL of DMF was added, and the solution was cooled in an ice-water bath. A solution of 9 mg DCC and 9 mg N-hydroxysuccinimide (NHS) in 0.5 mL of DMF was added upon stirring followed by a solution of 1 mg of 4-DMAP in 0.5 mL of DMF. The reaction mixture was kept in the ice-water bath for 5 min and then 18 hours at room temperature. The precipitated dicyclohexyl urea was filtered off and the solution was precipitated with acetone. After repeating this precipitation/filtration cycle three times the residue was dried under vacuum, dissolved in distilled water and dialyzed for 2 days against water. After lyophilization the conjugate was obtained (Yield: 175 mg). The conjugation of paclitaxel was confirmed by NMR and UV spectroscopy.

The disclosed polyglycerol-paclitaxel (GT) and polyglycerol-folate-paclitaxel (GFT) conjugates self-assemble to form nanoparticles in water due to the hydrophobic interaction between paclitaxel moieties. Thus, the methods disclosed herein can be used to prepare other nanoparticles self-assembled from conjugates including hydrophobic therapeutic agents. Those of skill in the art will be able to select suitable hydrophobic therapeutic agents from those disclosed herein for incorporation into the presently disclosed conjugate molecules.

### Example 10

### Synthesis of Labeled Conjugates

In this specific example, the conjugates included polyglycerol, folate, paclitaxel and the near infrared dye Cy5.5.

### Example 11

### Hydrophobic Drugs

In the foregoing examples, taxanes are used as an illustration of a hydrophobic drug that can be delivered using the hyperbranched polyglycerol nanoparticles disclosed herein. However, the use of taxanes is merely intended for illustration, and a variety of other hydrophobic drugs can also be conjugated to the nanoparticles.

The hydrophobicity of a compound may be measured by its solubility in water at room temperature (25 degrees C.) in units of grams per milliliter water. By this measure, the hydrophobic agents used for nanoparticle assembly are in the solubility range of 10 nanograms/ml (most hydrophobic such as cholesterol) to 1 milligram/ml (less hydrophobic such as doxorubicin). Aside from other intercalating agents, additional examples of hydrophobic anti-tumor drugs include intercalating agents such as cisplatin (cis-diammine dichloroplatinum II)

Among the hydrophobic drugs which may be conjugated to nanoparticles to help achieve self-assembly of the particles are the following classes of drugs, with particular listed examples of individual therapeutic agents:

Analgesics and anti-inflammatory agents: aloxiprin, auranofin, azapropazone, benorylate, diflunisal, etodolac, fenbufen, fenoprofen calcium, flurbiprofen, ibuprofen, indomethacin, ketoprofen, meclofenamic acid, mefenamic acid, nabumetone, naproxen, oxyphenbutazone, phenylbutazone, piroxicam, sulindac.
Anthelmintics: albendazole, bephenium hydroxynaphthoate, cambendazole, dichlorophen, ivermectin, mebendazole, oxamniquine, oxfendazole, oxantel embonate, praziquantel, pyrantel embonate, thiabendazole.
Anti-arrhythmic agents: amiodarone HCl, disopyramide, flecamide acetate, quinidine sulphate. Anti-bacterial agents: benethamine penicillin, cinoxacin, ciprofloxacin HCl, clarithromycin, clofazimine, cloxacillin, demeclocycline, doxycycline, erythromycin, ethionamide, imipenem, nalidixic acid, nitrofurantoin, rifampicin, spiramycin, sulphabenzamide, sulphadoxine, sulphamerazine, sulphacetamide, sulphadiazine, sulphafurazole, sulphamethoxazole, sulphapyridine, tetracycline, trimethoprim.
Anti-coagulants: dicoumarol, dipyridamole, nicoumalone, phenindione.
Anti-depressants: amoxapine, maprotiline HCl, mianserin HCL, nortriptyline HCl, trazodone HCL, trimipramine maleate.
Anti-diabetics: acetohexamide, chlorpropamide, glibenclamide, gliclazide, glipizide, tolazamide, tolbutamide.
Anti-epileptics: beclamide, carbamazepine, clonazepam, ethotoin, methoin, methsuximide, methylphenobarbitone, oxcarbazepine, paramethadione, phenacemide, phenobarbitone, phenyloin, phensuximide, primidone, sulthiame, valproic acid.
Anti-fungal agents: amphotericin, butoconazole nitrate, clotrimazole, econazole nitrate, fluconazole, flucytosine, griseofulvin, itraconazole, ketoconazole, miconazole, natamycin, nystatin, sulconazole nitrate, terbinafine HCl, terconazole, tioconazole, undecenoic acid.
Anti-gout agents: allopurinol, probenecid, sulphin-pyrazone.
Anti-hypertensive agents: amlodipine, benidipine, darodipine, dilitazem HCl, diazoxide, felodipine, guanabenz acetate, isradipine, minoxidil, nicardipine HCl, nifedipine, nimodipine, phenoxybenzamine HCl, prazosin HCL, reserpine, terazosin HCL.
Anti-malarials: amodiaquine, chloroquine, chlorproguanil HCl, halofantrine HCl, mefloquine HCl, proguanil HCl, pyrimethamine, quinine sulphate.
Anti-migraine agents: dihydroergotamine mesylate, ergotamine tartrate, methysergide maleate, pizotifen maleate, sumatriptan succinate.
Anti-muscarinic agents: atropine, benzhexol HCl, biperiden, ethopropazine HCl, hyoscyamine, mepenzolate bromide, oxyphencylcimine HCl, tropicamide.
Anti-neoplastic agents and Immunosuppressants: aminoglutethimide, amsacrine, azathioprine, busulphan, chlorambucil, cis-platin (cis-diammine dichloro platinum (II)), cyclosporin, dacarbazine, doxorubicin (Adriamycin), estramustine, etoposide, lomustine, melphalan, mercaptopurine, methotrexate, mitomycin, mitotane, mitozantrone, procarbazine HCl, tamoxifen citrate, testolactone.
Anti-protazoal agents: benznidazole, clioquinol, decoquinate, diiodohydroxyquinoline, diloxanide furoate, dinitolmide, furzolidone, metronidazole, nimorazole, nitrofurazone, ornidazole, tinidazole.
Anti-thyroid agents: carbimazole, propylthiouracil.
Anxiolytic, sedatives, hypnotics and neuroleptics: alprazolam, amylobarbitone, barbitone, bentazepam, bromazepam, bromperidol, brotizolam, butobarbitone, carbromal, chlordiazepoxide, chlormethiazole, chlorpromazine, clobazam, clotiazepam, clozapine, diazepam, droperidol, ethinamate, flunanisone, flunitrazepam, fluopromazine, flupenthixol decanoate, fluphenazine decanoate, flurazepam, haloperidol, lorazepam, lormetazepam, medazepam, meprobamate, methaqualone, midazolam, nitrazepam, oxazepam, pentobarbitone, perphenazine pimozide, prochlorperazine, sulpiride, temazepam, thioridazine, triazolam, zopiclone.
Beta Blockers: acebutolol, alprenolol, atenolol, labetalol, metoprolol, nadolol, oxprenolol, pindolol, propranolol.
Cardiac Inotropic agents: aminone, digitoxin, digoxin, enoximone, lanatoside C, medigoxin.
Corticosteroids: beclomethasone, betamethasone, budesonide, cortisone acetate, desoxymethasone, dexamethasone, fludrocortisone acetate, flunisolide, flucortolone, fluticasone propionate, hydrocortisone, methylprednisolone, prednisolone, prednisone, triamcinolone.
Diuretics: acetazolamide, amiloride, bendrofluazide, bumetanide, chlorothiazide, chlorthalidone, ethacrynic acid, frusemide, metolazone, spironolactone, triamterene.
Anti-Parkinsonian agents: bromocriptine mesylate, lysuride maleate.
Gastro-intestinal agents: bisacodyl, cimetidine, cisapride, diphenoxylate HCl, domperidone, famotidine, loperamide, mesalazine, nizatidine, omeprazole, ondansetron HCL, ranitidine HCl, sulphasalazine.
Histamine H,-Receptor Antagonists: acrivastine, astemizole, cinnarizine, cyclizine, cyproheptadine HCl, dimenhydrinate, flunarizine HCl, loratadine, meclozine HCl, oxatomide, terfenadine.
Lipid regulating agents: bezafibrate, clofibrate, fenofibrate, gemfibrozil, probucol.
Nitrates and other anti-anginal agents: amyl nitrate, glyceryl trinitrate, isosorbide dinitrate, isosorbide mononitrate, pentaerythritol tetranitrate.
Nutritional agents: betacarotene, vitamin A, vitamin B.sub.2, vitamin D, vitamin E, vitamin K.
Opioid analgesics: codeine, dextropropyoxyphene, diamorphine, dihydrocodeine, meptazinol, methadone, morphine, nalbuphine, pentazocine.
Sex hormones: clomiphene citrate, danazol, ethinyl estradiol, medroxyprogesterone acetate, mestranol, methyltestosterone, norethisterone, norgestrel, estradiol, conjugated oestrogens, progesterone, stanozolol, stibestrol, testosterone, tibolone.
Stimulants: amphetamine, dexamphetamine, dexfenfluramine, fenfluramine, mazindol.
Mixtures of hydrophobic drugs may, of course, be used where therapeutically effective.

In summary, a new class of nanoparticles is disclosed for tumor targeting, imaging and treatment by using hyperbranched polyglycerols as carriers for the therapeutic and imaging agents. The self-assembled multi-component nanoparticles demonstrate prolonged blood circulation and remarkably lower RES uptake while exhibiting excellent antitumor activity, facilitated by the balance of renal clearance and tumor accumulation. The targeted nanoparticles allow for the optical detection and treatment of very small tumors, offering new possibilities to treat tumor metastasis.

The compositions can include a hyperbranched polyglycerol polymer nanocarrier and a hydrophobic pharmaceutical agent, wherein the pharmaceutical agent is covalently bonded to the hyperbranched polymer nanocarrier to form a conjugate that self-assembles into a larger complex of conjugates. The pharmaceutical agent may be linked to the polyglycerol polymer by a linker. In some examples, the composition further includes a targeting agent covalently bonded to the conjugate in a sufficient amount to target the composition to a cellular target; the targeting agent may by linked to the polyglycerol polymer by a linker, such as a PEG linker The hyperbranched polymer has a degree of polymerization of 15-85 and a degree of branching of 0.5-0.9; and the pharmaceutical agent is a therapeutic agent or an imaging agent. In particular examples, the individual nanoparticle conjugates assemble into larger complexes of nanoparticle conjugates for transport through the circulatory system toward target cells.

In some examples, the composition has the formula Aₘ-X-Yₙ, wherein X represents the polyglycerol nanocarrier, A is a paclitaxel moiety and Y is a folate moiety, for example wherein n and m independently are from 1 to about 50; the hyperbranched polymer has an average molecular weight of at least about 250 daltons, about 250 to about 100,000 daltons, about 1,000 to about 80,000 daltons, or about 10,000 to about 50,000 daltons; and the pharmaceutical agent is a hydrophobic agent, such as an antitumor agent, such as a taxane, for example docetaxel or paclitaxel.

In certain examples, the pharmaceutical agent is covalently bonded to the hyperbranched polymer via a linker moiety, such as a succinate residue, an ethylene glycol moiety such as polyethylene glycol, or both. In other examples, the linker is (a) carboxylic acids from 2 to 10 carbons, for example, the linker derived from succinic anhydride that has 4 carbons, (b) amino acids from one to 10 amino acids in length; or (c) polyethylene glycols in the molecular weight range of 500 Daltons to 20 KDaltons.

In yet other examples, the nanoparticle composition includes a hydrophilic hyperbranched polymer, an antitumor agent covalently bonded to the hydrophilic polymer; and an antitumor agent noncovalently bound to the hydrophilic polymer. The antitumor agent covalently bonded to the hydrophilic polymer is bonded via a linker moiety, such as a succinate residue, polyethylene glycol, or both.

Also included within the disclosure is a nanoparticle composition produced according to the methods disclosed herein, and methods of treating a subject having a hyperproliferative disorder by administering to the subject the nanoparticle composition, thereby treating the subject. In particular examples, the subject has a condition such as breast cancer, bladder cancer, bone cancer, cervical cancer, colon cancer, central nervous system cancer, esophageal cancer, gall bladder cancer, gastrointestinal cancer, head and neck cancer, laryngeal cancer, leukemia, lung cancer, melanoma, ovarian cancer, prostate cancer or renal cancer.

It will be apparent to those skilled in the art that various modifications and variations can be made in the present compounds, compositions and methods without departing from the scope or spirit of the disclosure. Other embodiments of the compounds, compositions and methods will be apparent to those skilled in the art from consideration of the specification and practice of the procedures disclosed herein. It is intended that the specification and examples be considered as exemplary only, with a true scope and spirit of the invention being indicated by the following claims.

We claim:

1. A composition, comprising
a hyperbranched polyglycerol polymer nanocarrier and a hydrophobic pharmaceutical agent, wherein the pharmaceutical agent is covalently bonded to the hyperbranched polymer nanocarrier to form a conjugate that self-assembles into a larger complex of conjugates.

2. The composition of claim 1, further comprising a targeting agent covalently bonded to the conjugate in a sufficient amount to target the composition to a cellular target.

3. The composition of claim 1, wherein the hyperbranched polymer has a degree of polymerization of 15-85.

4. The composition of claim 1, wherein the hyperbranched polymer has a degree of branching of 0.5-0.9.

5. The composition of claim 1, wherein the pharmaceutical agent is a therapeutic agent or an imaging agent.

6. The composition of claims 1, wherein the composition has a formula
Aₘ-X-Yₙ,
X-Aₘ-Yₙ
or
X-Yₙ-Aₘ
wherein A represents a chemotherapeutic agent or an imaging agent; X represents the polyglycerol nanocarrier; and Y represents the targeting agent, and n and m independently are integers from 1 to about 500, such as from 5 to about 150.

7. The composition of claim 6, wherein n and m are integers of from 1 to 50, and the sum of m and n is from 10 to about 100, for example about 50.

8. The composition of claims 6, wherein n is greater than one, and the targeting agents are the same or different; and m is greater than one and the therapeutic agents are the same or different.

9. The composition of claim 7 having the formula Aₘ-X-Yₙ, wherein X represents the polyglycerol nanocarrier, A is a paclitaxel moiety and Y is a folate moiety, for example wherein n and m independently are from 1 to about 50.

10. The composition according to claim 1, wherein the hyperbranched polymer has an average molecular weight of at least about 250 daltons, about 250 to about 100,000 daltons, about 1,000 to about 80,000 daltons, or about 10,000 to about 50,000 daltons.

11. The composition of claim 1, wherein the pharmaceutical agent is a hydrophobic antitumor agent,

12. The composition of claim 1, wherein the antitumor agent is a taxane, for example docetaxel or paclitaxel.

13. The composition of claim 1, wherein the antitumor agent is docetaxel or paclitaxel.

14. The composition of claim 1, wherein the pharmaceutical agent is covalently bonded to the hyperbranched polymer via a linker moiety.

15. The composition of claim 14, wherein the linker moiety comprises polyethylene glycol.

16. The composition of claim 1, wherein the nanocarrier comprises paclitaxel and the folate.

17. The composition of claim 1, wherein the pharmaceutical agent covalently bonded to the hyperbranched polymer has the formula

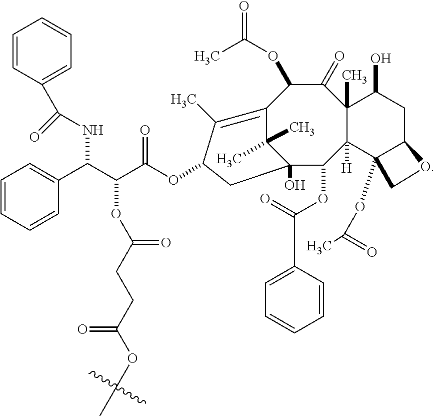

18. A nanoparticle composition, comprising:
a hydrophilic hyperbranched polymer;
an antitumor agent covalently bonded to the hydrophilic polymer; and an antitumor agent noncovalently bound to the hydrophilic polymer.

19. A method for treating a subject having a hyperproliferative disorder, comprising administering to the subject a composition according to claim 1, thereby treating the subject.

20. The method according to claim 19, wherein the subject has a condition comprising breast cancer, bladder cancer, bone cancer, cervical cancer, colon cancer, central nervous system cancer, esophageal cancer, gall bladder cancer, gastrointestinal cancer, head and neck cancer, laryngeal cancer, leukemia, lung cancer, melanoma, ovarian cancer, prostate cancer or renal cancer.
